(19) United States
(12) Patent Application Publication
(10) Pub. No.: US 2013/0197420 A1
(43) Pub. Date: Aug. 1, 2013

(54) NANOPOROUS MEMBRANES, DEVICES, AND METHODS FOR RESPIRATORY GAS EXCHANGE

(71) Applicants: William H. Fissell, IV, Brentwood, TN (US); Harihara Baskaran, Strongsville, OH (US); Shuvo Roy, San Francisco, CA (US); Ken Goldman, Olmsted Township, OH (US)

(72) Inventors: William H. Fissell, IV, Brentwood, TN (US); Harihara Baskaran, Strongsville, OH (US); Shuvo Roy, San Francisco, CA (US); Ken Goldman, Olmsted Township, OH (US)

(21) Appl. No.: 13/522,991

(22) PCT Filed: Jan. 19, 2011
(86) PCT No.: PCT/US11/21763

§ 371 Date: Nov. 5, 2012

Related U.S. Application Data
(60) Provisional application No. 61/296,160, filed on Jan. 19, 2010.
(60) Provisional application No. 61/431,262, filed on Jan. 10, 2011.

(51) Int. Cl. A61M 1/16

(52) U.S. Cl. CPC A61M 1/1698
USPC 604/6.16; 422/48

(57) ABSTRACT

One aspect of the present invention relates to a silicon nanoporous membrane for oxygenating blood. The nanoporous membrane includes a first major surface, a second major surface, and a plurality of pores extending between the first and second major surfaces. The first major surface is for contacting a gas. The second major surface is for contacting blood and is oppositely disposed from said first major surface. The first and second major surfaces define a membrane thickness. Each of the pores is defined by a length, a width, and a height. Each of the pores is separated by a uniform interpore distance.

### RELATED APPLICATION

The present application claims priority to U.S. Provisional Patent Application Ser. No. 61/296,160, filed Jan. 19, 2010, and U.S. Provisional Patent Application Ser. No. 61/431,262, filed Jan. 10, 2011, both of which are incorporated herein in their entireties.

### TECHNICAL FIELD

The present invention generally relates to membranes, devices, and methods for respiratory gas exchange, and more particularly to silicon nanoporous membranes with monodisperse pore size distributions, extracorporeal respiratory gas exchangers, and methods for respiratory gas exchange, such as oxygenating and/or removing carbon dioxide from blood.

### BACKGROUND OF THE INVENTION

Patients with injured or diseased lungs can be supported with supplemental oxygen, but face a grim choice when supplemental oxygen is unable to meet the patient's respiratory requirements. Mechanical ventilation (MV) via an endotracheal tube breeds its own set of problems, including ventilator-acquired pneumonia, further damage to diseased lungs, and the need for sedation, which interferes with eating and physical therapy. Patients receiving MV are susceptible to infection, malnutrition and deconditioning. When MV is unable to achieve adequate respiratory gas exchange, an artificial lung can be tried. Artificial lungs transmit oxygen to blood and remove carbon dioxide through a porous or woven polymer membrane. The membrane is connected to the patient through catheters inserted in large vessels, such as femoral veins and arteries, or the great vessels in the chest, and blood is pumped to the membrane at flow rates similar to cardiac output (4-6 L/min), a process called extracorporeal membrane oxygenation (ECMO).

Current ECMO therapy remains a highly invasive therapy due to the relatively large size of the oxygenator and pump mechanism; even with successful cannulation and gas exchange, patients are obligated to remain in an ICU setting, are generally unable to ambulate, and most often still require mechanical ventilation. In addition, ECMO therapy frequently requires intrathoracic access (post-cardiotomy support) or cannulation of the groin (femoral) vessels. This mandates bedrest and can lead to complications of vascular access, including limb ischemia from arterial cannulation and edema from venous outflow obstruction. Compartment syndrome and/or ischemia requiring amputation may result. Furthermore, traditional ECMO circuits require ongoing anticoagulation to prevent blood clotting of the oxygenator, which may cause bleeding diathesis and platelet consumption. Finally, the duration of ECMO is usually limited due to its implantation in immobile, critically ill, patients in the intensive care unit. Thus, the practical length of ECMO therapy is frequently limited due to the natural history of the patient's underlying illness or longer-term ICU complications, such as nosocomial infections, deconditioning, malnutrition, and pressure ulcers.

### SUMMARY OF THE INVENTION

According to one aspect of the present invention, a silicon nanoporous membrane for oxygenating and/or removing carbon dioxide from blood is provided. The nanoporous membrane comprises a first major surface, a second major surface, and a plurality of pores extending between the first and second major surfaces. The first major surface is for contacting a gas. The second major surface is for contacting blood and is oppositely disposed from said first major surface. The first and second major surfaces define a membrane thickness. Each of the pores is defined by a length, a width, and a height. Each of the pores is separated by a uniform interpore distance.

According to another aspect of the present invention, a portable extracorporeal respiratory gas exchanger is provided. The extracorporeal respiratory gas exchanger comprises a silicon nanoporous membrane, a housing, a first fluid passageway, a gas passageway, and a second fluid passageway. The nanoporous membrane comprises a first major surface, a second major surface, and a plurality of pores extending between the first and second major surfaces. The first major surface is for contacting a gas. The second major surface is for contacting blood and is oppositely disposed from said first major surface. The first and second major surfaces define a membrane thickness. Each of the pores is defined by a length, a width, and a height. Each of the pores is separated by a uniform interpore distance. The housing contains the nanoporous membrane. The first fluid passageway is configured to receive blood from a subject's vasculature and deliver blood to the second major surface of the nanoporous membrane. The gas passageway is configured to deliver the gas to the first major surface of the nanoporous membrane. The second fluid passageway is configured to remove oxygenated blood from the housing and deliver the oxygenated blood to the vasculature of the subject.

According to another aspect of the present invention, a method is provided for treating a respiratory disorder in a subject. One step of the method includes providing a portable extracorporeal respiratory gas exchanger. The extracorporeal respiratory gas exchanger comprises a silicon nanoporous membrane, a housing that contains the nanoporous membrane, a first fluid passageway, a second fluid passageway, and a gas passageway. The nanoporous membrane comprises oppositely disposed first and second major surfaces that define a membrane thickness, and a plurality of pores extending between the first and second major surfaces. Each of the pores is defined by a length, a width, and a height. Each of the pores is separated by a uniform interpore distance. Next, a vein and artery of the subject is connected to the first and second fluid passageways, respectively. A gas is then infused into the gas passageway at a pressure sufficient to ensure that the blood-gas phase interface is maintained at the second major surface of the nanoporous membrane. Blood flowing through the extracorporeal respiratory gas exchanger is oxygenated and delivered to the vasculature of the subject via the second fluid passageway.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1A is a perspective view showing a silicon nanoporous membrane constructed in accordance with one aspect of the present invention;

FIG. 1B is a cross-sectional view taken along Line 1B-1B in FIG. 1A;

FIG. 2A is a perspective view showing an alternative configuration of the silicon nanoporous membrane in FIGS. 1A-B;

FIG. 2B is a cross-sectional view taken along Line 2B-2B in FIG. 2A;

FIG. 3 is a perspective view showing an extracorporeal respiratory gas exchanger constructed in accordance with another aspect of the present invention;

FIGS. 4A-B are scanning electron micrographs (SEMs) showing highly uniform, slightly tapered (˜3°) 270 nm-wide micropores (FIG. 4A) and a high-density array of nanoporous membranes, each membrane containing over 2000 slit-shaped pores;

FIG. 5 is a schematic illustration showing the process flow for fabricating the nanoporous membrane in FIGS. 1A-B;

FIG. 6 is a schematic illustration showing the process flow for fabricating the nanoporous membrane in FIGS. 2A-B;

FIG. 7 is a plot showing high uniformity of oxidation growth (5-500 nm-thick) over a course of 25 distinct runs;

FIGS. 8A-B are a series of plots showing the effect of CO₂ saturation level (FIG. 8A) (at 10 SCCM carrier gas flow rate) and carrier gas flow rate (FIG. 8B) on CO₂ transport rate across nanoporous membranes (a recent microfabricated oxygenator's performance is shown as a dashed line);

FIG. 9 is a cross-sectional schematic view showing the geometry of the pores included in the nanoporous membrane in FIGS. 2A-B;

FIG. 10 is a schematic illustration showing gas transport in an individual tapered pore of FIG. 9;

FIG. 11 is a plot showing the effect of pore size on bubble point pressure for the pores included in the nanoporous membrane of FIGS. 1A-B;

FIG. 12 is a schematic illustration showing the gas-liquid system used to measure gas transport;

FIGS. 13A-B are a series of SEM micrographs of a microfabricated silicon nanopore membrane. FIG. 13A is a tilted top view showing the pore width (W) is 40 micrometers, and FIG. 13B is a side view showing the pore length (L) is 4.52 micrometers and pore height (h) is 13 nanometers;

FIG. 14A shows the molecular structure of the initiator silane and SBMA monomer;

FIG. 14B is an illustration showing the process of surface grafting via ATRP from silanized substrates;

FIG. 15 is an XPS survey scan spectra of bare silicon, silanized silicon, and polySBMA grafted silicon surfaces for 1.5 minutes and 1 hour;

FIG. 16 is a plot showing ellipsometric thickness of polySBMA on silicon as a function of polymerization time (error bars represent standard deviations among at least three measurements);

FIG. 17 is a plot showing the stability of polySBMA in PBS (pH 7.4, 5% CO₂ and 37° C.). The N₁ₛ/Si₂ₚ (solid squares) and S₂ₚ/Si₂ₚ (open squares) ratios were calculated from XPS measurements;

FIG. 18A is a plot showing the flow rate of water through nanoporous membranes at different pressure and after membrane coating with poly(SBMA);

FIG. 18B is a graph showing the calculated pore heights (or pore size) measured by the liquid permeability method for three nanoporous membrane chips (black bars) and after (white bars) coated with polySBMA;

FIGS. 19A-B are a series of plots showing fibrinogen adsorption from 1 mg/ml human fibrinogen (Fg) (FIG. 19A) and 10% PPP measured by ELISA (FIG. 19B). The results are compared with Fg adsorption on TCPS, PU, PTFE and PEG-silane coated silicon (optical densities were obtained by subtracting the negative control absorbance from the experimental values); and

FIG. 20 is a plot showing CO₂ removal rates across a silicon nanoporous membrane. Experiments were carried out in a transport chamber. 80% CO₂-saturated water was used as the liquid and pure N₂ was used as the gas. CO₂ transport from the liquid to the gas was measured as a function of N₂ gas pressure.

FIG. 22 demonstrates that as the sweep gas pressure is increased, the gas-liquid interface in the pores of the membrane moves towards the liquid side, which leads to enormous increase in the transport flux. The transition from mostly liquid-filled to mostly gas-filled pores occurs at a pressure of around 6 psig.

### DETAILED DESCRIPTION

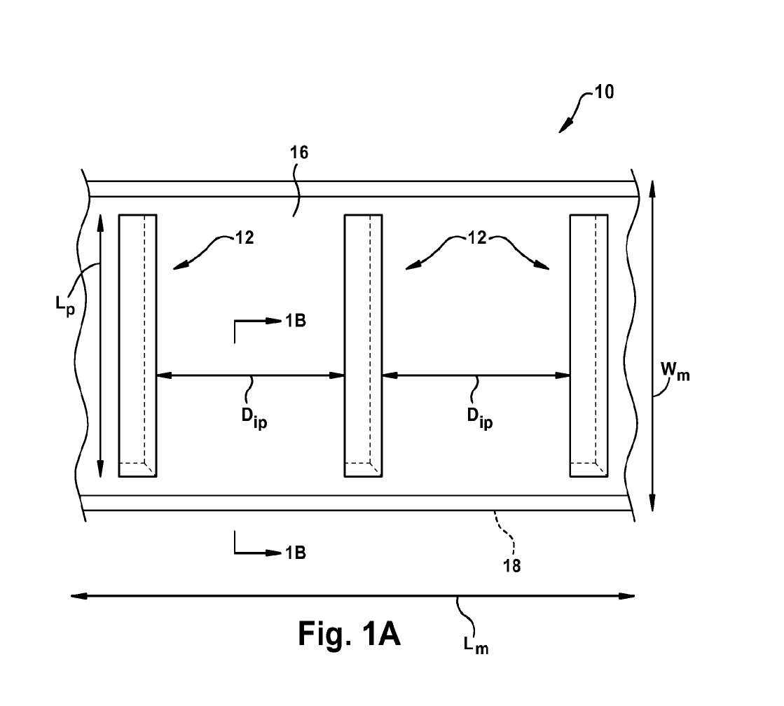

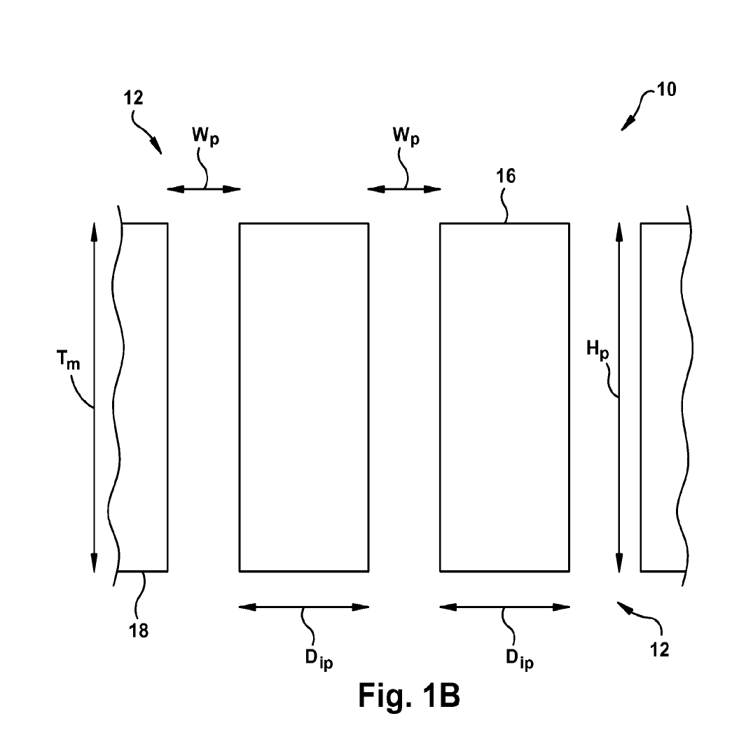

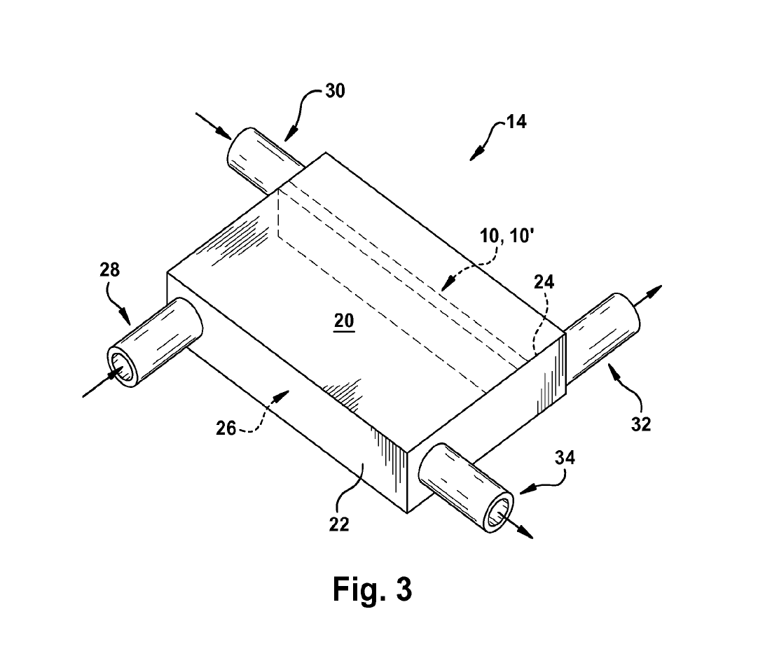

The present invention generally relates to membranes, devices, and methods for oxygenating and/or removing carbon dioxide from blood, and more particularly to silicon nanoporous membranes with monodisperse pore size distributions, extracorporeal respiratory gas exchangers, and methods for oxygenating and/or removing carbon dioxide blood using the same. As representative of one aspect of the present invention, FIGS. 1A-B illustrate a silicon nanoporous membrane 10 that includes a plurality of monodisperse pores 12, which permits differential pressure to control pore wetting and thus gas transport. That the pores 12 and 12′ are the same size and thus has the same bubble point, allows transmembrane pressure to control the meniscus position identically in all pores, controlling pore wetting without gas embolization. As discussed in more detail below, the nanoporous membranes 10 of the present invention can advantageously withstand exceptionally high transmembrane pressures through the pores 12 without gas embolization. Consequently, the nanoporous membranes 10 of the present invention enable the development of minimally invasive and portable extracorporeal respiratory gas exchanger s14 (FIG. 3) that have substantially greater (10-25 times) gas exchange area per unit volume compared to conventional membrane oxygenators.

As shown in FIGS. 1A-B, one aspect of the present invention includes a silicon nanoporous membrane 10. The nanoporous membrane 10 comprises a first major surface 16 for contacting a gas (e.g., oxygen), and a second major surface 18 for contacting a fluid, such as blood or plasma. The first and second major surfaces 16 and 18 of the nanoporous membrane 10 are oppositely disposed from one another and together define a membrane thickness Tₘ. The nanoporous membrane 10 can have a membrane thickness Tₘ of about 0.1 micrometers to about 50 micrometers. In one example of the present invention, the nanoporous membrane 10 can have a membrane thickness Tₘ of about 1 micrometer to about 5 micrometers. In another example of the present invention, the nanoporous membrane 10 can have a membrane thickness Tₘ of about 4 micrometers. The membrane thickness Tₘ can be uniform or non-uniform. A non-uniform membrane thickness Tₘ, for example, may exhibit increased strength as compared to a uniform membrane thickness Tₘ.

The nanoporous membrane 10 includes a length Lₘ and a width Wₘ. The length Lₘ and the width Wₘ of the nanoporous membrane 10 can be varied depending upon the particular application of the nanoporous membrane; however, the length Lₘ and the width Wₘ can generally range from about 0.1 micrometer to about 1000 micrometers or more. For example, the nanoporous membrane 10 can have a rectangular shape and include a length Lₘ of about 10 micrometers to about 500 micrometers, and width Wₘ of about 10 micrometers to about 500 micrometers. It will be appreciated that the nanoporous membrane 10 can have other shapes as well, such as square, ovoid, circular, etc. As shown in FIGS. 1A-B, the nanoporous membrane 10 has a flattened, sheet-like configuration. The flattened, sheet-like configuration of the nanoporous membrane 10 can minimize pressure drop across the nanoporous membrane when used for extracorporeal membrane oxygenation (ECMO), for example.

The nanoporous membrane 10 of the present invention can be made of any one or combination of biocompatible materials suitable for use in oxygenating a fluid, such as blood or oxygen. Examples of materials include silicon, as well as coated silicon materials (described below). More particularly, materials that may be used to form the nanoporous membrane 10 can include any one or combination of silicon, polysilicon, silicon carbide, silicon dioxide, PMMA, SU-8, and PTFE. Other possible materials include metals (e.g., titanium) and ceramics (e.g., silica or silicon nitride).

In one example of the present invention, the nanoporous membrane 10 is made of silicon.

The nanoporous membrane 10 additionally includes a plurality of pores 12 extending between the first and second major surfaces 16 and 18. Each of the pores 12 is defined by a length Lₚ, a width Wₚ, and a height Hₚ that can be equal to or about equal to the membrane thickness Tₘ. The length L, width Wₘ and height Hₚ of each of the pores 12 is the same throughout the nanoporous membrane 10. Each of the pores 12 is separated from one another by an interpore distance Dᵢₚ. The interpore distance Dᵢₚ can be uniform or different between pores 12 and can be, for example, less than about 5 micrometers (e.g., less than about 3 micrometers). The nanoporous membrane 10 can include any number of pores 12, ranging from just two pores up to a million or more pores.

The monodisperse pore size distribution—or, the fact that the dimensions (e.g., Lₚ, Wₘ and Hₚ) of the pores 12 are uniform—is advantageous for several reasons. For example, it is known that the leading cause of device failure in ECMO is pore wetting. The nanoporous membrane 10 of the present invention (when used during ECMO) separates the liquid phase of blood from the gas phase of the sweep gas (e.g., oxygen) to prevent a subject from bleeding into the extracorporeal respiratory gas exchanger 14 (FIG. 3) and prevent gas emboli from entering into the subject's blood. The pressure at which sweep gas embolizes into the subject's blood is referred to as the “bubble point” of an ECMO membrane, and is set by the dimensions of the largest pore in the membrane (e.g., the bigger the largest pore, the lower the pressure at which bubbles form in the blood). For polymer membranes, log-normal pore size distributions are common, which means that for any membrane there are many pores substantially larger than the average pore size of the membrane. To control bubble point, commercial practice has been to engineer the mean pore size of the membrane so small that there are so few pores so big as to threaten a gas embolus. The very small pore size limits membrane gas transfer, however, and the broad pore size distribution still limits the sweep gas pressure that may be safely used. Thus, unlike conventional membranes (e.g., polymer membranes) used for ECMO, the nanoporous membrane 10 of the present invention advantageously includes monodisperse pore size distribution such that each of the pores 12 has the same bubble point. Consequently, membrane failure through pore wetting is prevented or mitigated.

As shown in FIGS. 1A-B, each of the pores 12 is generally slit-shaped and has a symmetrical cross-sectional profile (e.g., defined by the height Hₚ and width Wₚ). For example, each of the pores 12 has a rectangular cross-sectional profile. It will be appreciated that the pores 12 can have other cross-sectional profiles, such as square, circular, ovoid, elliptical, etc. Additionally, it will be appreciated that the pores 12 can have other shapes besides a slit-shaped configuration. The length Lₚ of each of the pores 12 can be about 3 micrometers to about 100 micrometers or more. In one example of the present invention, the length Lₚ of each of the pores 12 can be about 5 micrometers to about 45 micrometers. Additionally, the width Wₚ of each of the pores 12 is at least about 10 micrometers and, for example, at least about 0.5 micrometers to about 11 micrometer.

The extraordinarily uniform membrane pore size and shape provides at least three advantages over conventional ECMO membranes: (1) the monodisperse pores maximize pore size (and thus gas transfer) while also maximizing bubble point; (2) a high bubble point allows sweep gas pressure to maintain the blood-gas phase interface at the blood side (i.e., the second major surface 18) of the nanoporous membrane 10; and (3) the flat sheet design of the nanoporous membrane minimizes pressure drop when used during ECMO, allowing pumpless ECMO.

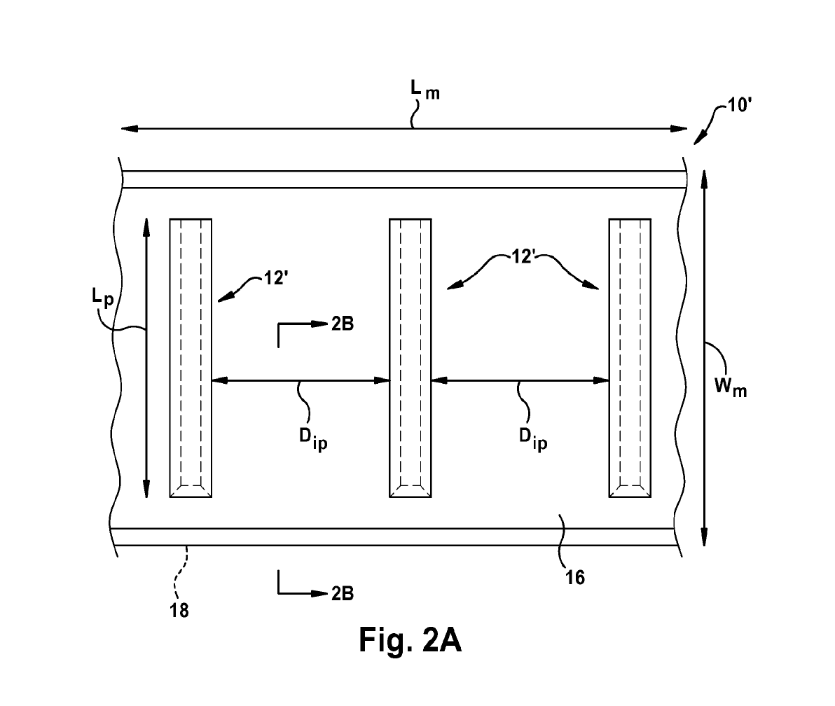

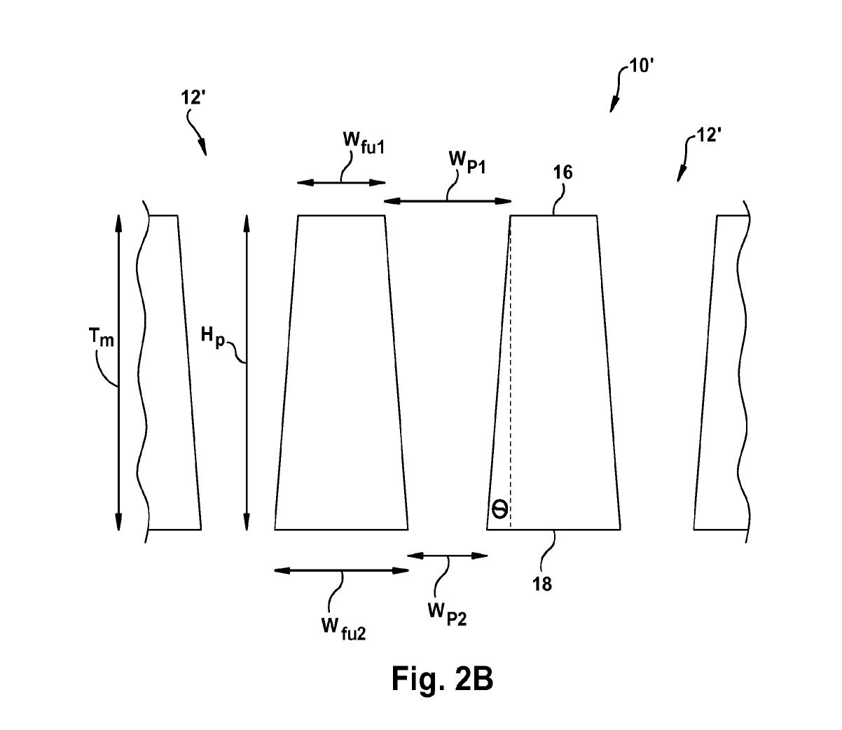

Another aspect of the present invention is illustrated in FIGS. 2A-B and includes a silicon nanoporous membrane 10′ that is identical to the nanoporous membrane shown in FIGS. 1A-B, except that the nanoporous membrane 10′ includes a plurality of pores 12′ having asymmetrical cross-sectional profiles. More particularly, each of the pores 12′ has an asymmetric tapered cross-sectional profile that enhances pressure control of the phase interface, in turn making it easier to maintain an equilibrium position with sweep gas pressure along. As shown in FIG. 2A, each of the pores 12′ is slit-shaped; however, it will be appreciated that other shapes are possible.

Each of the pores 12′ (FIGS. 2A-B) is defined by a height H, that is equal to or about equal to the membrane thickness Tₘ, a length Lₚ, a first width Wₚ₁, and a second width Wₚ₂. As shown in FIG. 2B, the first width Wₚ₁ is greater than the second width Wₚ₂. The area efficiency of gas transport is determined by the ratio of the second width Wₚ₂ and a final unopened width W_{fu2}. This ratio is a simple geometric function of the taper angle θ and the membrane thickness Tₘ, resulting in Wₚ₂/W_{fu2}=(Wₚ₁−2Tₘ·tan θ)/(W_{fu1}+2Tₘ·tan θ). Gas transport membrane area efficiency is inversely reduced as the tapered angle θ increased for a fixed width W_{fu1} and membrane thickness Tₘ by reducing the ratio of Wₚ₂ to W_{fu2}. To increase the ratio to 1:1 (50%), for example, a smaller value of W_{fu1} is needed. This can be accomplished using e-beam, nanoimprint, or argon fluoride based nanolithography. It will be appreciated that the taper angle θ can also be varied as need for each of the pores 12′. For example, the taper angle θ can be varied from about 10° to less than 900. Advantageously, the asymmetric (i.e., tapered) cross-sectional profile of the pores 12′ enhances pressure control of the phase interface, making it easier to maintain an equilibrium position with sweep gas pressure alone.

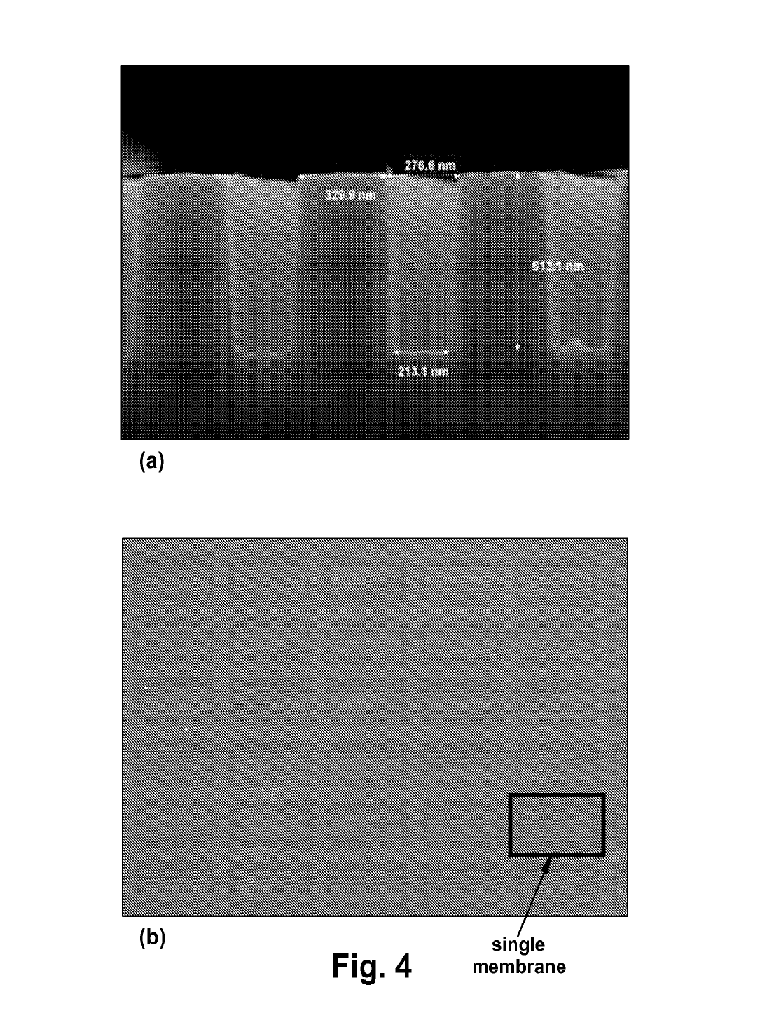

It will be appreciated that, depending upon the particular application of the present invention, two or more nanoporous membranes 10 and/or 10′ can be arranged in parallel or in series to form a sandwich-like or sheet-like configuration, respectively. When arranged in series, for example, each of the nanoporous membranes 10 and/or 10′ can be arranged in an end-to-end configuration to form a sheet comprising multiple nanoporous membranes, such as the high-density array of nanoporous membranes (each containing over 2000 slit-shaped pores) as shown in (FIG. 4B). As described in more detail below, such nanoporous membrane 10 and/or 10′ configurations can be used in medical devices during ECMO, for example.

It will also be appreciated that all or only a portion of the nanoporous membrane 10 and/or 10′ can be treated (e.g., coated) with one or more biocompatible materials to prevent or mitigate biofouling. The portion(s) of the nanoporous membrane 10 and/or 10′ treated with the one or more biocompatible materials creates a low fouling surface that resists adsorption of not only protein, but also cell adhesion, adhesion of bacteria and other microorganisms, and biofilm formation. Suitable biocompatible materials useful for treating the nanoporous membrane 10 and/or 10′ include zwitterionic materials, which are electronically neutral materials that typically include equal amounts of positive charges and negative charges. In one example of the present invention, the biocompatible material used to treat all or only a portion of the nanoporous membrane 10 and/or 10′ can include sulfobetaine materials, such as poly(sulfobetaine methacrylate) (polySBMA) that include sulfate negative charges and ammonium positive charges. Other biocompatible materials that may be used alone or in combination with zwitterionic materials can include PEG, heparin, and PVAm.

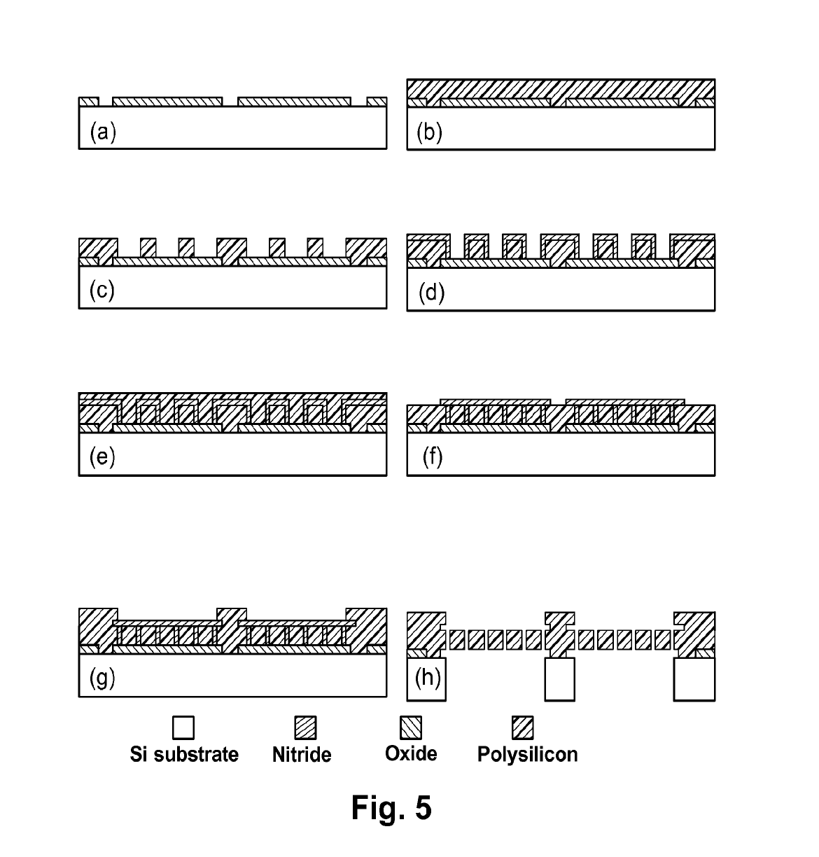

The pores 12 and 12′ of present invention can be created by micro-machining (referred to as “nanofabrication”) techniques. Micromachining is a process that includes photolithography, such as that used in the semiconductor industry, to remove material from, or to add material to, a substrate. The nanoporous membrane 10 illustrated in FIGS. 1A-B can be manufactured as shown in FIG. 5, for example. As shown in FIG. 5, the starting material can be a conventional silicon wafer. First, at step (a) polysilicon anchors can be etched into about a 0.5 micrometer-thick nitride layer using standard photolithography and etching techniques. At step (b), a layer of polysilicon of about 0.5-1.0 micrometer thickness can be deposited. Then, electron beam nanolithography and reactive ion etching can be used to pattern the polysilicon layer (step (c)). At step (d), an oxide layer of about 50-100 nanometer thickness can be grown on the polysilicon layer to define the pore size. Next, a polysilicon layer of about 750 nanometer thickness is deposited at step (e), thereby filling in the patterned gaps. At step (f), dry etching can be used to planarize the front surface. Also, an oxide layer of about 0.5 micrometer thickness can be deposited and patterned, forming anchors. At step (g), a polysilicon layer of about 1.5 micrometer thickness can be deposited and patterned over the anchors etched in step (f). The oxide in step (f) is an etch-stop layer for patterning of this polysilicon layer, which anchors the second polysilicon layer to the first polysilicon layer. Next, the silicon substrate can be etched anisotropically at step (h) using deep reactive ion etching (DRIE), stopping on the nitride layer. The buried oxide can act as an etch stop for the DRIE. Lastly, the nanoporous membrane 10 can be released and the pores opened using hydrofluoric acid.

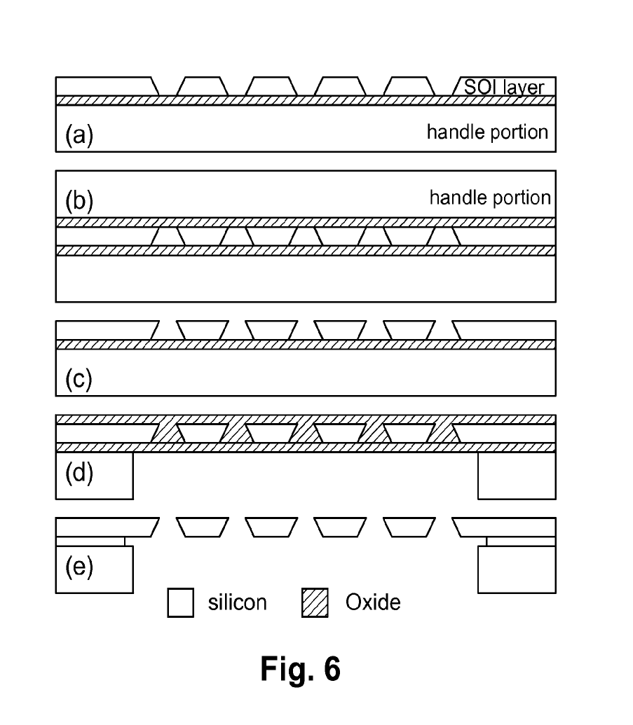

In another example of the present invention, the nanoporous membrane 10′ (FIGS. 2A-B) can be formed according to the microfabrication process illustrated in FIG. 6. For example, Photomasks designed using layout software can be used in the membrane manufacturing process and silicon on insulator (SOI) wafers may be used as the starting material. The first process step can be to pattern the SOI layer into about 500-1000 nm pores using nanolithography and reactive ion etching at step (a). The etching process can be tuned to provide the tapered profile needed for optimum bubble-point control and gas transport performance. After etching the SOI layer to obtain the needed taper, the patterned SOI wafer can be flipped and bonded to a double-side polished (DSP) wafer using silicon fusion bonding at step (b). Note that an oxide layer may be used in the fusion bonding, thus resulting in the second oxide layer. This bonding can transpose (flip) the tapered pattern, orienting the taper in the correct manner (top side blood flow). Alternatively, a retro-graded etch process, which can be difficult to control, could be used to form the tapered pores, thus eliminating the need for transposing. Next, at step (c), the SOI wafer handle portion can be removed using DRIE. The buried oxide can act as an etch stop for the DRIE process. Hydrofluoric acid may then be used to remove the oxide etch stop layer. This removal process can also remove a small amount of the underlying oxide that resulted from the fusion bonding in step (b). Subsequently, a protective coat of low-stress oxide can be deposited on the wafer to prevent scratching and to solidify the remaining oxide layer. Backside cavities may then be etched in the wafers using DRIE and the oxide layer as an etch stop (step (d)). At step (e), the oxide layer can finally be removed using hydrofluoric acid.

Another aspect of the present invention includes a portable extracorporeal respiratory gas exchanger 14 for oxygenating and/or removing carbon dioxide from blood. As shown in FIG. 3, the extracorporeal respiratory gas exchanger 14 generally comprises a housing 20 that contains one or more nanoporous membranes 10 and/or 10′ having a flat sheet configuration, which minimizes pressure drop within the exchanger and thereby allows pumpless ECMO. As described below, the extracorporeal respiratory gas exchanger 14 can be connected to a subject using upper extremity vessels, which permits minimally invasive or even ambulatory ECMO. It will be appreciated that the nanopous membrane(s) 10 and/or 10′ comprising the extracorporeal respiratory gas exchanger 14 can be optimized and further assembled into a minimally invasive cartridge. The nanoporous membrane(s) 10 and/or 10′ contained in the housing 20 separate(s) the liquid phase of blood from the gas phase of the sweep gas (e.g., oxygen) to prevent the subject from bleeding into the extracorporeal respiratory gas exchanger and prevent gas emboli from entering into the subject's blood. The housing 20 can include any one or combination of the nanoporous membranes 10 and/or 10′ described above.

The housing 20 generally comprises an outer surface 22 and inner surface 24 that defines a compartment 26. The housing 20 can be made of any desired material. Where the housing 20 is used on or in a subject, for example, the housing can be made of or coated with a biocompatible material. Although the housing 20 shown in FIG. 3 has a rectangular shape, it will be appreciated that the housing can have any shape suitable for accommodating one or more nanoporous membranes 10 and/or 10′. Likewise, the compartment 26 can have any appropriate shape and configuration such that the compartment can accommodate one or more nanoporous membranes 10 and/or 10′. The nanoporous membrane(s) 10 and/or 10′ can form two or more compartments (not shown in detail) within the housing 20, each of which is separated by a nanoporous membrane, such that each compartment is in fluid communication with the other compartment only by means of the pores 12 and/or 12′ within the nanoporous membrane(s).

The extracorporeal respiratory gas exchanger 14 also includes a mechanism for permitting entry into the housing 20 (e.g., a first compartment) of a deoxygenated fluid (e.g., venous blood) from the vasculature of a subject, a mechanism for permitting entry of a gas (e.g., oxygen) into the compartment 26 (e.g., a second compartment), and a mechanism for permitting exit of an oxygenated fluid (e.g., oxygenated blood) into the vasculature of a subject. For example, the extracorporeal respiratory gas exchanger 14 can include a first fluid passageway 28 that is configured to receive venous blood from a subject's vasculature and deliver the venous blood to the second major surface 18 of a nanoporous membrane 10 and/or 10′. Additionally, the extracorporeal respiratory gas exchanger 14 can include a gas passageway 30 configured to deliver a gas (e.g., oxygen) to the first major surface 16 of a nanoporous membrane 10 and/or 10′, and a second fluid passageway 32 configured to remove oxygenated blood from the compartment 26 into the vasculature of a subject. The extracorporeal respiratory gas exchanger 14 can additionally or optionally include a second gas passageway 34 that is configured to remove at least some of the gas from the compartment 26.

In one example of the present invention, the extracorporeal respiratory gas exchanger 14 can have a cross-flow oxygenator design, which allows for separate blood and gas manifolds (not shown) and simplifies device construction. Such a configuration can consist often separate 500 micrometer blood flow channels, for example. Two MEMS chips (not shown), sandwiched back-to-back, at a total layer thickness of about 1000 micrometers, can separate the blood flow channels and create the ventilating gas flow path. The blood flow channels can be about 50 mm², providing a total blood contact surface of about 500 cm². A side port (not shown) can optionally or additionally be connected to the gas passageway for monitoring gas inlet pressure.

Compared to conventional respiratory gas exchangers having similar packing densities, the well-defined uniform nanoscale pores 12 and 12′ of the present invention have substantially greater (e.g., 10-25 times) gas exchange per unit area. The parallel-plate design of the extracorporeal respiratory gas exchanger 14 leads to very low pressure drop in the device, which, as noted above, allows pumpless implementation of the extracorporeal respiratory gas exchanger. Additionally, smaller packaging due to highly efficient gas transport also provides an extracorporeal respiratory gas exchanger 14 that enhances blood-membrane contacting efficiency, which is an important mechanism of gas transport in respiratory gas exchangers.

As noted above, the primary cause of failure of conventional gas exchangers (e.g., oxygenators) is gradual membrane failure due to pore wetting. Pore wetting can be controlled in a few ways. The most common approach involves modifying the surface chemistry of the pores. For example, a hydrophobic surface tends to exclude water and keep the pores dry. However, hydrophobic surfaces promote protein binding at the phase interface, altering the contact angle at the pore surface, which essentially makes the pore hydrophilic and wicking water (and more protein) into the pore. In theory, one could exclude water or plasma from the pore by pressurizing the sweep gas to oppose fluid intrusion into the pore; although, high pressures are required for a hydrophilic material. Such a technique cannot be used in conventional oxygenators, however, due to the polydispersity of the polymer membrane pores; that is, the pressure needed to exclude water from the most numerous small pores will exceed the bubble point of the membrane dictated by the fewer, larger pores.

Unlike conventional gas exchangers, the extracorporeal respiratory gas exchanger 14 of the present invention includes at least one nanoporous membrane 10 and/or 10′ with monodisperse pore size distributions, i.e., there is no “largest pore”. Since a nanoporous membrane 10 and/or 10′ with monodisperse pores 12 and/or 12′ has the same bubble point for all pores, the position of the liquid-gas phase interface within the pores is uniform across the membrane surface. Advantageously, this allows the extracorporeal respiratory gas exchanger 14 to be operated at the sweep gas pressure required to oppose fluid water intrusion into the pores 12 and/or 12′ and, thus, the uniform pore size of the nanoporous membrane facilitates an unconventional approach for prolonging membrane life by preventing the nanoporous membrane 10 and/or 10′ from “wetting out”.

Another aspect of the present invention includes a method for treating a respiratory disorder in a subject. Respiratory disorders treatable by the present invention can include both infection-induced and non-infection-induced diseases and dysfunctions of the respiratory system. For example, respiratory disorders treatable by the present invention can include chronic lung disease and acute lung injury. Subjects suffering from chronic lung disease are in need of a bridge-to-transplant device that will sustain their life until lung transplant can occur, while acute lung injury subjects require a bridge-to-recovery device that will relieve the respiratory burden on the lungs and promote a return of lung function. Unlike conventional methods for treating chronic lung disease and acute lung injury, which use extracorporeal membrane oxygenators and mechanical ventilators that are traumatic to the body, the method of the present invention uses an extracorporeal respiratory gas exchanger 14 capable of providing the needed bridge-to-recovery or bridge-to-transplant without further stressing already fragile subjects.

One step of the method includes providing a portable extracorporeal respiratory gas exchanger 14. The extracorporeal respiratory gas exchanger 14 can be similar or identical to the one described above. For example, the extracorporeal respiratory gas exchanger 14 can have a compact, pumpless design and include at least one nanoporous membrane 10 and/or 10′, a housing 20 containing the at least one nanoporous membrane, a first fluid passageway 28 configured to receive deoxygenated blood from the subject's vasculature, a gas passageway 30 configured to deliver a gas (e.g., oxygen) to the at least one nanoporous membrane, and a second fluid passageway 32 configured to remove oxygenated blood from the compartment 26 of the extracorporeal respiratory gas exchanger.

The extracorporeal respiratory gas exchanger 14 is connected to upper extremity vessels (not shown) of the subject, such as the axillary artery and the cephalic vein. For example, the first fluid passageway 28 can be surgically connected to the cephalic vein of the subject so that deoxygenated blood is delivered to the second major surface 18 of the at least one nanoporous membrane 10 and/or 10′. Additionally, the second fluid passageway 32 can be surgically connected to the axillary artery. Next, a gas, such as pure oxygen can be infused into the gas passageway 30 and thus into contact with the first major surface 16 of the at least one nanoporous membrane 10 and/or 10′. The oxygen can be infused into the gas passageway 30 at a pressure sufficient to ensure that the blood-oxygen phase interface is maintained at the second major surface 18 of the at least one nanoporous membrane 10 and/or 10′. It will be appreciated that the extracorporeal respiratory gas exchanger 14 can be surgically connected to any other artery or vein, depending upon the particular medical needs of the subject.

As the oxygen contacts the blood, there are three serially occurring transport processes that constitute the overall transport mechanism in the pores 12 and/or 12′ of the at least one membrane 10 and/or 10′. Any oxygen molecule that is transported from the gas-phase to the blood-phase is first transported from the flowing gas stream into the pore 12 and/or 12′ primarily through convection, after which it is transported in the pore through primarily a diffusion mechanism, and finally to the plasma of blood flowing on the opposite side in a counter-current manner. Once in plasma, the oxygen molecule is transported by diffusion into the red blood cells where it rapidly reacts with hemoglobin, its carrier in blood. Carbon dioxide follows a reverse path. A key difference is that carbon dioxide is stored in blood primarily in a bicarbonate form and to a smaller extent as a hemoglobin bound form. Bicarbonate ions combine with protons in the presence of carbonic anhydrase, a highly efficient enzyme in red blood cells, to release carbon dioxide. The above process is enhanced by the oxygen-hemoglobin reaction, which leads to the release of protons, an effect known as Haldane effect.

After properly connecting the extracorporeal respiratory gas exchanger 14 to the subject, blood can flow continuously through the extracorporeal respiratory gas exchanger so that deoxygenated blood is continuously oxygenated and oxygenated blood is continuously delivered to the vasculature of the subject. Advantageously, the method of the present invention augments the respiratory capacity of damaged lungs, and thus can improve care, in at least two ways. First, partial support of the subject with chronic lung disease awaiting transplant can delay or eliminate the need for mechanical ventilation, thereby allowing the subject to eat normally and maintain physical conditioning so that subjects are transplanted when they are medically at their best, rather than at their worst. Second, the use of a minimally invasive extracorporeal respiratory gas exchanger 14 can lower the threshold at which ECMO can be offered to subjects with acute lung injury, facilitating lung sparing ventilation and potentially improving outcomes in acute lung injury. For example, blood flows in the axillary artery and the cephalic vein could support up to a liter per minute of blood flow to the extracorporeal respiratory gas exchanger and 100-200 ml/min of respiratory gas exchange, or more than half the subject's metabolic requirements.

The following examples are for the purpose of illustration only and are not intended to limit the scope of the claims, which are appended hereto.

### Example 1

### Silicon Microporous and Nanoporous Membranes are Manufactured With High Precision

Nanoporous membranes with monodisperse pores have been developed and prototyped using an innovative process based on MEMS (micro electro mechanical systems) technology. MEMS devices are unique in that they utilize not only the electrical properties of semiconductor materials, but also rely heavily on the mechanical performance and structuring of such materials. Such mechanical features are used to create movable structures to create sensors and micromanipulators, for example.

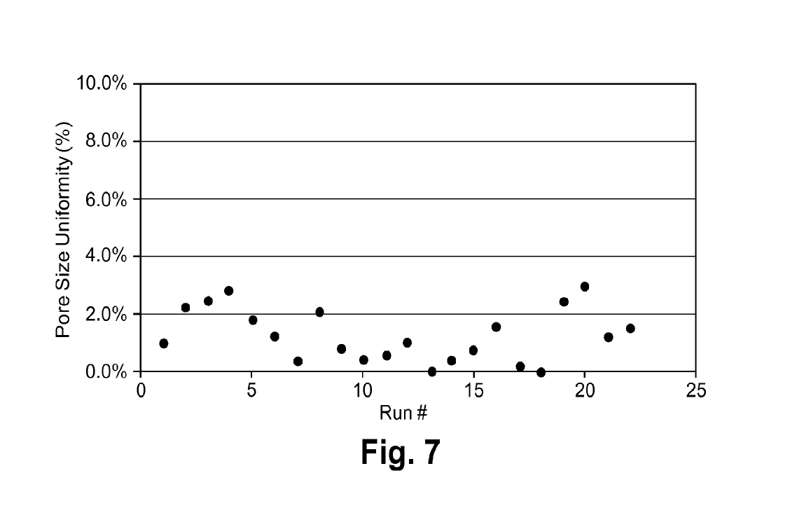

The manufacturing process of the present invention uses advanced nanolithography and thermal processing to establish the critical submicron pore size and density. FIGS. 4A-B show scanning electron microscopy (SEM) images of highly-uniform nanopores (FIG. 4A) and of a high-density array of filtration membranes (FIG. 4B). Pores sizes have been readily varied between 5-500 nm, and we have successfully controlled pore sizes with <3% variation over the course of nearly 25 distinct processing runs, as shown in FIG. 7.

Silicon Nanopore Membranes can Withstand the Rigors of Packaging and Surgical Procedures

In a surgical planning study for an implantable hemofilter (not shown), a polycarbonate housing was designed by SimuTech, Inc. (Rochester, N.Y.) and prototyped at the Cleveland Clinic (Cleveland, Ohio). A 500 nm pore size silicon membrane, manufactured by HCubed, Inc. (Olmstead Falls, Ohio), was coated with PEG and secured in the housing with a silicone gasket. A 46 kg Yorkshire breed pig was sedated with ketamine and 2% isoflurane and a right open nephrectomy was performed. Polytetrafluoroethylene (PTFE) grafts were sutured to the remnant renal artery and vein and secured to the housing with silk sutures. The animal was heparinized with 1000 U unfractionated heparin followed by a 500 U/hour infusion. Stable blood and ultrafiltration flow rates were maintained over the 2.5 hour planned surgery, except during an inadvertent kinking of the arterial graft during closing of the animal, which was quickly reversed. At time of sacrifice no thrombus on the membrane or within the housing. The membrane remained intact after PEG coating, mounting within the housing, during surgical handling, and during direct contact with arterial blood.

Carbon Dioxide Transport is Highly Efficient Across Silicon Nanoporous Membranes

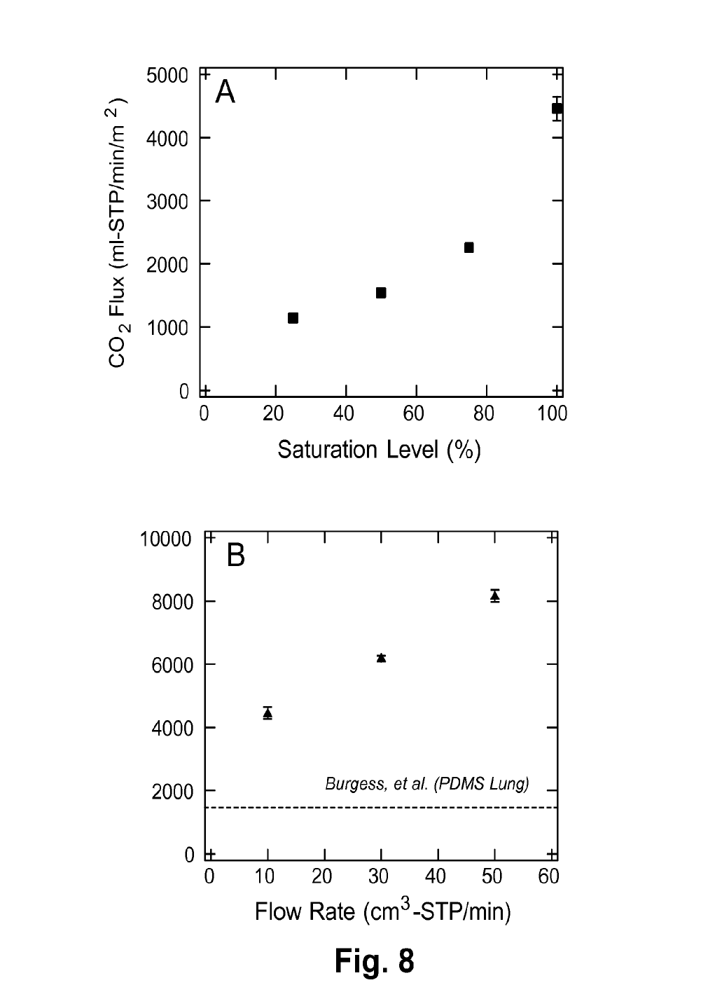

To determine transport efficiency of our membranes, we carried out carbon dioxide transport measurements. In these experiments, water samples with carbon dioxide at various levels of saturation at 1 atm and 298° K. were pumped on one side of the membrane in a dual chamber transport device. A carrier gas, N₂, flowed on the other side of the membrane. We assessed the effect of CO₂ equilibration levels and carrier gas flow (10-50 SCCM) on the transport rate of CO₂. Our results (FIG. 8A) show that we are able to achieve very high CO₂ transport rates in the device, a level that is considerably greater than published values for a novel microfabricated PDMS silicone oxygenator (horizontal line in FIG. 8B) (Burgess K A et al., Biomedical Microdevices 11:117-127, 2009).

Computational Fluid Dynamics Predicts Low Pressure Drop and Low Blood Trauma

The preliminary computational fluid dynamics (CFD) studies focused the oxygenator blood flow path design. The specific goals were to: (1) assess the flow uniformity amongst the blood channels; and (2) identify any regions of flow recirculation or stasis within the oxygenator. Improving the flow uniformity amongst the blood channels increases the oxygenator's overall gas transfer effectiveness (i.e., reduces shunting), and minimizing areas of stasis reduces the potential for thrombus formation. Future CFD models will include the blood and gas side flow paths to predict overall oxygenator performance.

Analysis methodology: the commercial software packages, DesignModeler and CFX, from ANSYS (ANSYS Inc., Canonsburg, Pa.) were used to create the model and perform the CFD simulation. The CFD solutions were performed on a Dell 8-processor workstation with 32 GB of RAM.

Oxygenator model: a cross-flow oxygenator design (not shown) is envisioned. This design allows for separate blood and gas manifolds and simplifies the device construction. The baseline oxygenator design consists of 10 separate 500 micrometer blood flow channels. Two MEMS chips, sandwiched back-to-back at a total layer thickness of 1000 micrometers, separate the blood flow channels and create the ventilating gas flow path. The blood flow channels are 50 mm square, providing a total blood contact surface area of 500 cm². A side port is connected to the gas inlet connection for monitoring gas inlet pressure.

Blood path CFD model: in this initial work, a three-dimensional CFD model was created of the oxygenator blood flow path. Hexahedral elements were used to create the meshes in the blood channels and channel entrance/exit regions. In the geometrically complex manifold regions, tetrahedral/prism elements were used, with inflated prism elements for capturing the boundary layer flows along the manifold walls. The blood was modeled at 37° C. and incorporated a Cross non-Newtonian viscosity model (Cross N M, J Colloid Sci. 20:417-437, 1965). The inlet blood flow rate was set at 300 ml/min, a value near the expected upper range for the animal-designed oxygenator. Due to the low blood velocities and small flow path dimensions, the entire oxygenator was modeled under laminar flow conditions. Steady state flow conditions were also assumed for these initial analyses.

CFD results summary: the flow within the oxygenator was controlled by the 500 micrometer thick blood channels. The flow resistance provided by these blood channels was 1.38 mmHg, which agrees well with the theoretical Poiseuille parallel plates pressure drop of 1.30 mmHg. This resistance was sufficient to provide very good flow uniformity (peak exit velocity±2%) amongst all flow channels. Residence time contour analysis (not shown) revealed the slightly faster center region flow but overall good blood washout through the device. Very low fluid shear stress values (<2 Pa) were found throughout the oxygenator, levels significantly below threshold hemolysis and platelet lysis values (Goubergrits L., Expert Rev Med Devices 3:527-531, 2006).

### Example 2

### Establish Microfabrication Techniques to Create a Novel Membrane with Highly Uniform Tapered Pores to Facilitate Control of Pore Wetting

The leading cause of device failure in extracorporeal membrane oxygenation (ECMO), pore wetting, may be controlled by maintaining the liquid-gas phase transition at the blood side of the membrane. This can be achieved with sweep gas pressure rather than surface chemistry, but doing so risks gas embolus if a pressure transient disturbs the equilibrium position of the meniscus. An asymmetric tapered pore enhances pressure control of the phase interface, making it easier to maintain an equilibrium position with sweep gas pressure alone. To enhance control of pore shape and asymmetry, our existing microfabrication protocols are optimized.

In order to create tapered pores, refinements to our previously established microfabrication process are made. These refinements include adjusting etch parameters to obtain higher pore taper (asymmetry) and the use of silicon fusion bonding to transpose the pore geometry. A detailed step-by-step, cross-sectional process flow diagram for micropore oxygenation membranes is shown in FIG. 6. Photomasks are used in the membrane manufacturing process, and silicon on insulator (SOI) wafers are used as the starting material. The first process step is to pattern the SOI layer into 500-1000 nm pores using nanolithography and reactive ion etching (FIG. 6(a)). The etching process is tuned to provide the tapered profile needed for optimum bubble-point control and gas transport performance. After etching the SOT layer to obtain the needed taper, the patterned SOI wafer is flipped and bonded to a double-side polished (DSP) wafer using silicon fusion bonding (FIG. 6(b)). Note that an oxide layer is used in the fusion bonding, thus resulting in the second oxide layer. This bonding transposes (flips) the tapered pattern, orienting the taper in the correct manner (top side blood flow). Next (FIG. 6(c)), the SOI wafer handle portion is removed using deep reactive ion etching (DRIE). The buried oxide acts as an etch stop for the DRIE process. Hydrofluoric acid is then used to remove the oxide etch stop layer this removal process will also remove a small amount of the underlying oxide that resulted from the fusion bonding in step (b). Subsequently, a protective coat of low-stress oxide is deposited on the wafer to prevent scratching and to solidify the remaining oxide layer. Backside cavities are etched in the wafers using DRIE and the oxide layer as an etch stop (FIG. 6(d)). Finally, the oxide layer is removed using hydrofluoric acid (FIG. 6(e)).

Use Transport Models and In Vitro Experiments to Optimize Pore Shape And Size in the Nanopore Membranes

Pore optimization in polymer membranes is challenging as pore characteristics are governed by the thermodynamics and chemistry of the polymer melt. Silicon nanotechnology allows one to refine pore geometry in response to transport model predictions. Transport models are developed to predict gas exchange through tapered pores, carry out small scale in vitro gas-water and gas-blood experiments, and use the results to optimize the pore geometry of the membrane and operating parameters of the oxygenator.

### Gas Transport Modeling of Membranes

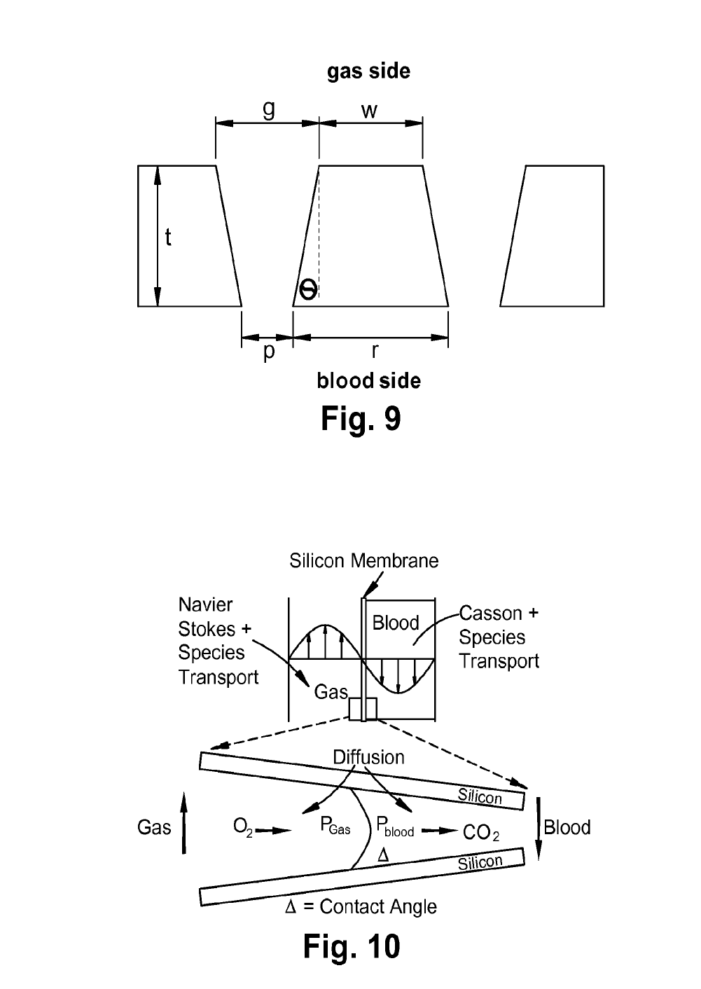

A schematic illustration of a rectangular-shaped nanoslit is shown in FIG. 9. The performance of the device depends on the transport efficiency of these slits. Gas transport in silicon nanoporous membranes using a multi-scale modeling approach is used. In this approach, gas transport is modeled in individual nanoslits. Gas transport rates are determined in nanoslits as a function of local variables and physiochemical properties of fluids. These include pore size, inlet flow rate for liquid/blood, and gas pressures. Utilizing these functional dependencies, overall gas transport in the entire device is modeled, which contains millions of nanoslits.

In the nanoslits, there are three serially occurring transport processes that constitute the overall transport mechanism. Any oxygen molecule that is transported from the gas-phase to the blood-phase is first transported from the flowing gas stream into the nanoslit primarily through convection, after which it is transported in the nanoslit through primarily a diffusion mechanism and finally to the plasma of blood flowing on the opposite side in a counter-current manner. Once in plasma, the oxygen molecule is transported by diffusion into the red blood cells where it rapidly reacts with hemoglobin, its carrier in blood. Carbon dioxide follows a reverse path. A key difference is that carbon dioxide is stored in blood primarily in a bicarbonate form and to a smaller extent as a hemoglobin bound form. Bicarbonate ions combine with protons in the presence of carbonic anhydrase, a highly efficient enzyme in red blood cells, to release carbon dioxide. The above process is enhanced by the oxygen-hemoglobin reaction, which leads to the release of protons, an effect known as Haldane effect.

One consideration is the location of the gas-plasma interface in the nanoslit. This determines whether diffusion within the pore occurs in liquid phase (slow), or in gas phase, which is substantially faster. The location depends on the interfacial tension between the gas and plasma phases, the contact angle between the silicon surface and the plasma phase, the geometry of the nanoslit (FIG. 10), and the pressure difference between the phases. In addition, the transport fluxes of the species can also impact the location of the interface. In a tapered geometry, the pore size varies as a function of depth position from the mouth of the pore, and this allows for higher pressures on the gas side leading to gas filled nanoslits.

In the model, local equilibrium is assumed and the Young-Laplace equation is used. To solve this equation, the pressure variations within the device as a function of location are needed. This information is obtained by numerical simulations using commercially available software (CFX, ANSYS, Canonsburg, Pa.). The primary boundary condition involves the use of three-phase contact angle. Solution of the equation allows the position of the gas-liquid interface in the nanoporous slit to be determined.

Referring to FIG. 10, numerical simulations are performed to obtain pressure variations within the device. The Navier-Stokes equation is used for momentum balance in the bulk fluid flow of the gas, and a Cross non-Newtonian viscosity model is used for the blood. For these streams, the transport equation is used for species conservation (oxygen, carbon dioxide). The boundary condition on the nanoslit, which is common to both streams, matches the flux through the nanoslit and the concentration, taking into account the solubility relationship between gas and blood plasma. In the nanoslit, the fluids are considered to be static, so only diffusion is modeled as shown, for oxygen and carbon dioxide. The gas-blood interface has a contact angle, θ, and the pressure difference across the interface is P_{gas}−P_{blood}. The position of the interface is a solution of its force balance both statically and for a moving contact line in the tapered slit. The contact angle is varied to simulate protein deposition. From the velocity profiles, species conservation laws in gas and liquid phases are also solved. For the bulk transport in the liquid and gas phases, Fick's law of diffusion is considered. In blood phase, equilibria for oxygen-hemoglobin and carbon dioxide-bicarbonate reactions is considered. One consideration in solving species conservation laws is the flux boundary condition at the membrane. Solution of Navier-Stokes equations and subsequently the Young-Laplace equation determine the location of the phase-interface in the tapered slit, which is then used to determine the overall flux across the membrane through both phases in the tapered slit. Fick's law of diffusion is utilized with no convection to model the flux.

### Gas-Gas Transport Testing

To isolate and understand the mechanism of transport in the membrane, it is useful to study species transport from gas phase across the membrane. Further, these experiments allow us to obtain information regarding structural anomalies in the membranes before we subject them to gas-liquid/gas-blood experiments. In this task, the optimized membranes are tested using those manufactured above. Transport flux of carbon dioxide and oxygen (mol/cm²-s) is measured for the optimized pore size. As a control, previously tested membranes are measured, in which data was presented in Example 1. Nitrogen is used as the carrier gas. The semiconductor grade gases are first passed through a 0.2 μm filter. Mass flow controllers are used to adjust the flow rates of the gases from static to 1000 sccm in increments of 100 sccm. This is used to determine the effect flow rate has on the transmembrane flux. A mass spectrometer (Dycor, Pittsburgh, Pa.) downstream is used to detect the gas permeation rates through the membrane. Data is collected, tabulated, and compared to the modeling results. Scanning electron microscopy is used to determine exact membrane geometries for model comparisons. Refinements to the model are made if necessary.

### Bubble Point Testing

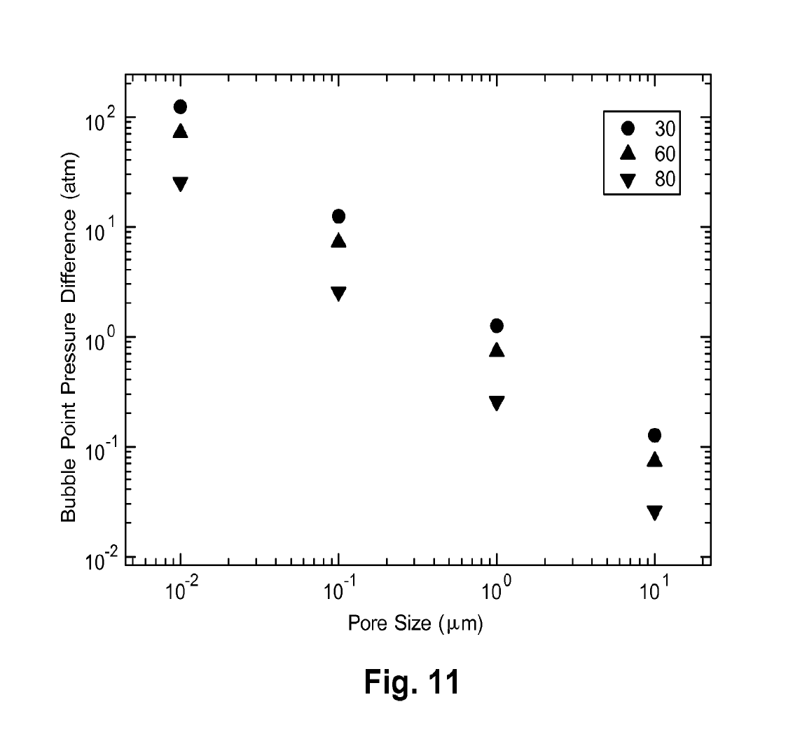

Various membranes fabricated in Example 1 are tested. For pores ranging in size from 10 nm-500 nm, gas and liquid pressure differential (Pgas−Pblood) values are tested that range from 0 to 2 atm. Membranes with tapered geometry are used; straight geometry membranes are used as controls. Testing is done up to the theoretical limit or the breaking point whichever is smaller. In addition, the membranes are tested after they are used in the animal experiments for bubble point to determine protein deposition and its effect on contact angle. FIG. 11 shows a graph of bubble point pressure as a function of pore size, based on the solution to the Young-Laplace equation for straight pore geometry. A similar graph is developed for tapered nanoslits and used as guidance in our testing. Data is plotted and compared to the theoretical values. As before, the contact angles are varied to simulate protein deposition for tapered pores of various angles.

### Gas-Liquid and Gas-Blood Transport Testing

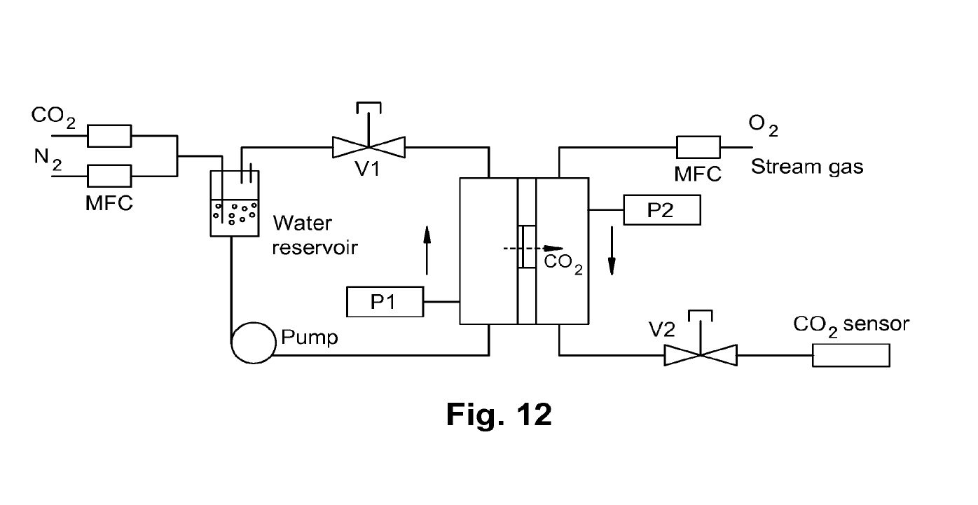

Here, the same membranes as described above are tested in a liquid-gas system if they are not damaged. Otherwise, new chips will be used. FIG. 12 shows a schematic of the mock used to test the membrane for gas-liquid transport. The loop is instrumented with pressure transducers (PX61 Omega Engineering, Stamford, Conn.) on both sides of the membrane (P1 and P2) and a mass spectrometer downstream of the gas side. Mass flow controllers (MFC) regulate the sparging of Nitrogen and CO₂ into the deionized (DI) water reservoir and oxygen as stream gas across the membrane. Pinch valves V1 and V2 are used to regulate pressures on each chamber and to set the differential pressure across the membrane. The DI water is circulated across the membrane using a peristaltic laboratory pump (Cole Parmer, Vernon Hills, Ill.). Similarly, O₂ transport is tested using mass spectrometer and a blood-gas analyzer for measuring oxygen flux.

Finally, the membrane chips are tested using fresh, citrated bovine blood. Citrate chelates calcium, an important cofactor in blood coagulation. Citrate prevents clotting caused by other parts of the blood loop during testing. Chips are coated with PEG prior to testing. This test is conducted over 24-96 hour periods. A similar setup as the one used for gas-water system except that a conventional membrane oxygenator (Affinity NT, Medtronic, Inc., Minneapolis, Minn.), instead of a sparger, is used for controlling blood CO₂ levels in blood. One concern is hemolysis of the bovine blood from red cell aging or due to trauma from the roller pumps be used, which are not specifically designed for blood perfusion. Depending on hemolysis as determined by interval postcartridge sampling (every four hour), membranes may be perfused in a recirculating fashion with blood or with a single-pass design. Blood gas analysis of pre- and postcartridge blood is conducted regularly every four hours for the first sixteen hours and then every eight thereafter. Oxygen partial pressure, CO₂ partial pressure, hemoglobin content, and hemoglobin saturation are measured with a clinical blood gas analyzer and total oxygen content calculated. The pre-post differential s used to calculate O₂ and CO₂ transport by the membrane at a spectrum of blood and stream gas flow rates, and to determine the performance of the membrane chip. Post testing, the membrane chip is analyzed for platelet adsorption using scanning electron microscopy (SEM) and ELISA.

Demonstrate 30 ml/min Nonventilatory Respiratory Support by the Silicon Nanoporous Membrane Oxygenator in a Hypercarbic/Hypoxemic Large Animal Model

Safety is of paramount importance if a minimally invasive oxygenator might be an alternative to mechanical ventilation. Computational fluid dynamics (CFD) is used to optimize blood path design for blood trauma and thrombosis, and test implementations of the cartridge in a large animal model of respiratory failure to validate predictions of blood trauma, thrombosis, gas transport, and a preliminary examination of safety at elevated sweep gas pressure.

Three-dimensional CFD models of the oxygenator blood and gas flow paths are created. As done previously, these 3D geometries are meshed using elements well-suited to resolve the internal surface geometry and resulting flow fields. ANSYS-CFX software is again used for the CFD simulations that is performed using the SimuTech workstation computer cluster. The majority of the simulations planned are performed under laminar, steady state flow conditions, but the effect of time-dependent (i.e., transient) flow effects are considered. Scalar values for the oxygen and carbon dioxide concentration in the blood are added to predict the gas exchanged between the blood and ventilating gas. The gas transfer methods described by Baker are incorporated into the CFD model to relate the gas partial pressures to their concentration in the blood (Baker D., Modeling of hollow-fiber blood-gas exchange devices: University of Minnesota, 1989). The membranes are modeled as a porous media with the gas transfer resistances based upon single MEMS chip test results. The Cross non-Newtonian blood viscosity model is again used throughout these analyses. To establish design criteria to avoid cell lysis and thrombosis formation, CFD analyses is used to predict the blood residence time and blood shear stress levels throughout the device. Several cell lysis predictive models are explored, including the threshold model, the cumulative injury “power-law” model, and an Eulerian control-volume based method (Cross M M, cited above; Goubergrits L, cited above; Giersiepen M et al., Int J Artif Organs 13:300-6, 1990; Bludszuweit C., Artif Organs 19:590-6, 1995; Paul R., Artif Organs 27:517-29, 2003; Garon A et al., Artif Organs 28:1016-25, 2004; Fill B et al., 54(2):1A-67A, 2008). Qualitative expressions are used to identify regions with increased potential for thrombus formation (e.g., low values of shear stress/residence time).

### CFD Program Structure

Initial CFD studies are dedicated to performing screening CFD analyses of 3-4 oxygenator design concepts. The impact of key design variables, such as blood rate, the number, aspect ratio, and height of the blood channels and manifold orientation/design on the overall device performance are predicted. Preliminary analyses modeling the gas transfer through the permeable MEMS chip and into the ventilating gas is performed. In addition to performing grid sensitivity studies using the Roache method, the CFD results are correlated with theoretical Poiseuille flow and in vitro experimental data to establish their validity (Roache P J, J Fluids Engr. 116:405-13, 1994). The preferred oxygenator flow path design from initial simulations is refined after the first animal experiments to provide preclinical data regarding clotting and blood trauma. Transient flow effects, simulating a blood pressure pulse, are studied along with the inclusion of blood trauma models for predicting cell lysis and thrombus formation (Cross M M, cited above; Goubergrits L, cited above; Giersiepen M et al., Int J Artif Organs 13:300-6, 1990; Bludszuweit C., Artif Organs 19:590-6, 1995; Paul R., Artif Organs 27:517-29, 2003; Garon A et al., Artif Organs 28:1016-25, 2004; Fill B et al., 54(2):1A-67A, 2008; Sagi R et al., Annals of Biomedical Engr. 35:493-504, 2007).

### Oxygenator Prototype Fabrication

A closely integrated effort between the flow path guiding CFD studies and the engineering design and prototype fabrication is planned. Blood compatible polymeric materials are machined or cast to create the structure for the oxygenator prototypes. Thin gaskets are used to seal and separate the blood and gas flow paths, allowing for interchangeability of membranes as needed. The flow path designs created in ANSYS DesignModeler and/or SolidWorks (Dassault Systemes SolidWorks Corp., Concord, Mass.) are transferred to the Cleveland Clinic's rapid prototyping equipment or CNC machining centers (MasterCAM, CNC Software, Inc., Tolland, Conn.). Direct connectivity between all these software packages allows for rapid transfer, manipulation, and fabrication of the oxygenator geometry.

### Demonstrate 30 Ml/Min Respiratory Gas Exchange By the Extracorporeal Oxygenator in a Hypercarbic/Hypoxemic Animal Model

First, safety of prototype oxygenators, including thombosis, hemolysis, membrane reliability, and gas emboli is assessed prior to scale-up. Second, predictions regarding respiratory gas delivery, and, in particular, the ability of stream gas pressure to prevent pore wetting over a range of blood flows and sweep gas pressures is examined.

The oxygenator cartridge design developed by SimuTech is manufactured at the Cleveland Clinic's Prototype Core. Membranes from H-Cubed are surface-modified with PEG and mounted in the cartridge. Oxygenators are tested in a hypoxemic/hypercarbic animal model to validate blood trauma and transport data in a live animal. Up to twenty 40-50 kg Yorkshire breed pigs are used in five groups of experiments.

A common set of procedures is used for all experiments: animals are anesthetized with ketamine and isoflurane. A right or left neck paramedian incision is used to approach the carotid artery and internal jugular vein, which is cannulated directly using pediatric cannulas sutured to the surrounding tissues. A second arterial catheter for blood gas analysis is placed as well. A conventional continuous dialysis machine and tubing set is used to pump blood from the carotid, through the oxygenator, and back to the jugular. The oxygenator cartridge is pressurized with sweep gas using mass flow controllers (as described above) prior to priming the circuit with saline. The animal is heparinized and the extracorporeal circuit connected. Hemoglobin and platelet counts are monitored before and after exposure to the circuit. Measurements described below are conducted over 4-5 hours and the animal euthanized with Beuthanasia solution. After euthanasia, samples of lung tissue are harvested for histologic examination for embolus.

The first group of animals (n=5) is used to evaluate the patency and thrombgenicity of the extracorporeal cartridge and assess acute hemolysis by the blood circuit. Results are passed back to SimuTech to refine cartridge design.

The second group of animals (n=5) is used to validate surface modification and fixation strategies for the silicon membranes in the final cartridge geometry. Membrane chips are extracted from the device after blood exposure and examined by light microscopy, SEM, and immunofluorescence for protein and platelet adsorption and thrombosis.

The third group of animals (n=5) is used to assess oxygen delivery and carbon dioxide removal by a cartridge containing chips with straight-sidewall pores. The fraction of inspired oxygen and the minute volume is varied to simulate hypoxemic and hypercarbic respiratory failure. Blood gas analysis of pre-filter, postfilter, and systemic samples is obtained to assess extracorporeal gas exchange.

The fourth group of animals (n=5) is used to assess oxygen delivery and carbon dioxide removal by a cartridge containing chips with tapered-sidewall pores. The fraction of inspired oxygen and the minute volume is varied to simulate hypoxemic and hypercarbic respiratory failure. Blood gas analysis of pre-filter, postfilter, and systemic samples are obtained to assess extracorporeal gas exchange. The effect of sweep gas pressure on oxygen transport and CO₂ removal in cartridges with tapered pores is then assessed. Sweep gas pressure is varied stepwise from atmospheric pressure up to the bubble point of the membrane. CO₂ flux is measured at each pressure using exhausted sweep gas. Data is fitted to transport models (from above) to estimate pore wetting. Pressures are be cycled to explore the possibility of forcing liquid out of a wetted pore with sweep gas pressure.

### Example 3

### Materials and Methods

### Materials and Synthesis

3-Aminopropyltrimethoxysilane was purchased from United Chemical Technologies (Bristol, Pa., USA). Triethylamine, α-bromoisobutyryl bromide (BIBB, 98%), tetrahydrofuran (THF, H-PLC grade), bicyclohexyl, copper(I) bromide (CuBr, 99.999%), copper(II) bromide (CuBr2, 99.999%), 2,2′-bipyridyl (BPY, 99%), [2-(methacryloyloxy)ethyl]dimethyl-(3-sulfopropyl)ammonium hydroxide (SBMA, 97%), phosphate-buffered saline (PBS, 0.01 M phosphate buffer, 0.137 M sodium chloride, 0.0027 M potassium chloride, pH 7.4) were purchased from Sigma-Aldrich. Water used in the experiments was purified using a Millipore water purification system (Billerica, Mass., USA) with a resistivity of 18.2 MΩ·cm.

The ATRP initiator, 2-bromo-2-methyl-N-3-[(trimethoxysilyl)propyl]-propanamide (BrTMOS), was synthesized in our own laboratory according to the literature (Z. Zhang et al., Langmuir 22, 10072, 2006). Briefly, 3-aminopropyltrimethoxysilane (10 mmol) was mixed with triethylamine (10 mmol) in dried THF (50 ml). BIBB (12 mmol) was added drop-wise into the solution for 30 min with stirring under a bubbling stream of nitrogen. Reaction was allowed to continue overnight (12+ h) under nitrogen protection. The precipitate was filtered off using a frit funnel. After the removal of the solvent by a rotary evaporator, the product was re-dissolved in hexane (20 ml). The solvent was then removed using the rotary evaporator, and the resulting colorless oil was dried in a vacuum oven overnight with a yield of 90%. ¹H-NMR (300 MHz, CHCl₃): δ6.85 (s, 1H, NH), 3.55 (s, 9H, SiOCH₃), 3.25 (t, 2H, CtH₂N), 1.95 (s, 6H, CH₃), 1.65 (m, CH₂, 2H), 0.65 (t, 2H, SiCH₂).

### Pretreatment of Silicon Surfaces and Fabrication of Silicon Nanopore Membranes (SNMs)

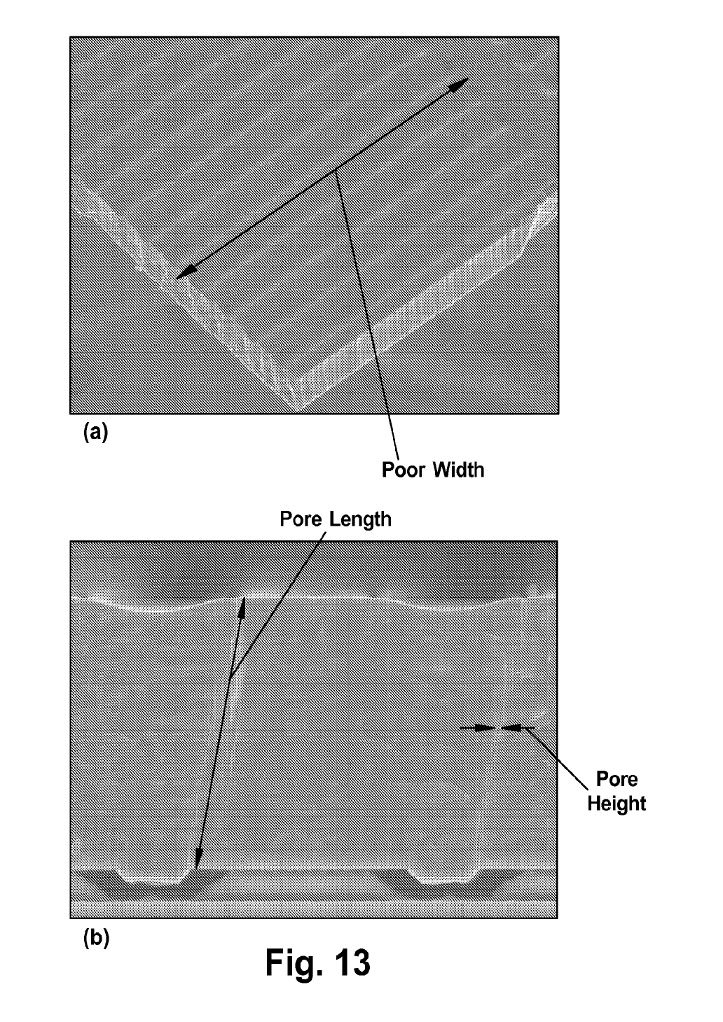

Prime grade, double side polished, {100}-oriented, n-type, silicon (Si) wafers were diced into 1 cm×1 cm chips and cleaned using the conventional ‘piranha’ cleaning procedure. Briefly, sample chips were cleaned by immersion in a solution of H₂O/ethanol (1:1, v/v) for 2 h, then thoroughly rinsed with deionized (DI) water, dried and placed in a freshly prepared ‘piranha’ solution (30% H₂O₂/96% H₂SO₄, 1:3) for 20 min. Caution: piranha solution is a strong oxidant and reacts violently with organic substances. Nanopore filtration membranes with monodisperse pore size distributions have been prototyped from silicon substrates by an innovative process based on MEMS technology (Lopez C A et al., Biomaterials 27, 3075, 2006; Leoni L et al., Biomed. Microdev. 4, 131, 2002). The process uses the controlled growth of a thin sacrificial SiO₂ (oxide) layer to define the critical submicron pore size of the filter. The oxide is etched away in the final step of the fabrication process to leave behind arrays of parallel 40-μm-long slit pores (FIGS. 13A-B) on 1 cm×1 cm chips.

### Preparation of PolySBMA on Silane-Coated Surfaces

The substrates were rinsed, dried and immediately placed in an anhydrous bicyclohexyl solution of BrTMOS (1%, v/v). The substrates were left in the solution for 2 h, after which they were removed from the solution, rinsed with chloroform and DI water, and dried in air.

Substrates with immobilized initiators were placed in a flask under nitrogen protection and sealed with rubber septum stoppers. SBMA monomer (1.06 g, 3.8 mmol) and BPY (312 mg, 2 mmol) were dissolved in a degassed solution (DI water/methanol=1:1 (v/v), 10 ml). CuBr₂ (67 mg, 0.3 mmol) was added to the solution and the mixture was degassed for 20 min. CuBr (143 mg, 1.0 mmol) was then added, and the polymerization solution was then transferred to the flask using a syringe under nitrogen protection. After reaction for different reaction times, the substrates were removed and rinsed with ethanol and water. The samples were kept in water overnight.

### Surface Characterization

XPS spectra were obtained on a PHI VersaProbe XPS Microprobe (Physical Electronics, Chanhassen, Minn., USA). An aluminum Kα monochromatized X-ray source is used to stimulate photoemission. The energy of the emitted electrons is measured with a hemispherical energy analyzer at pass energy of 117.4 eV. The binding energy (BE) scale is referenced by setting the peak maximum in the C1s spectrum to 285 eV. Spectra are collected with the analyzer at 45° with respect to the surface normal of the sample. Typical pressure in the analysis chamber during spectral acquisition is 10-9 Torr. Data analysis software from PHI MultiPack is used to calculate elemental compositions from the peak areas.

Contact angle measurements were carried out with a Rame-Hart contact angle goniometer by the sessile drop method in ambient conditions.

SEM analysis of the samples was performed using a Hitachi S4500 Field-Emission Scanning Electron Microscope (FESEM) equipped with a Noran XEDS (X-ray energy-dispersive spectrometry) system. Surfaces were examined both at low magnification and high magnification. For FESEM examination, the SNM chip was sectioned along the pore to visualize the inner surface of the pores. An accelerating voltage of 5 kV was used, which was suitable for the semi-conductive silicon surface of the membrane.

Film thickness of polySBMA on silicon wafers was collected with a triple wavelength Rudolph AutoEL-IV ellipsometer (Rudolph Research, Flanders, N.J., USA). The system automatically calculates ellipsometric parameters, thickness and index. An external PC with the customized software converts the measured delta (Δ, the relative phase change) and phi (r, the relative amplitude change) introduced by reflection from the surface into thickness and refractive index. A refractive index of 1.45 was assigned to the initiator and polymer layers.

### Hydraulic Permeability Characterization

SNM chips were positioned in an ultrafiltration cell and hydraulic permeability to gas and liquid was measured as previously described (Fissell W H et al., Am. J. Physiol. Renal Physiol. 293, F1209, 207; Fissell W H et al., J. Am. Soc. Nephrol. 13, 602A, 2002). Briefly, SNM were mounted in a custom-built ultrafiltration cell and flushed with carbon dioxide to exclude nitrogen. The feed and permeate sides of the membrane were wetted with DI water, and the feed side was pressurized with compressed air. Transmembrane pressures were adjusted to 0.50, 1.00, 1.50 and 2.00 psi. Movement of the fluid-air meniscus within a calibrated syringe on the permeate side was timed and volumetric flows were calculated.

### Protein Adsorption

An enzyme-linked immunosorbent assay (ELISA) was used to measure adsorption of fibrinogen and 10% platelet-poor plasma (PPP) to surfaces covered with polySBMA. Blood was obtained from a healthy volunteer and mixed with sodium citrate (0.38% final concentration). PPP was isolated by centrifugation at 3000×g for 15 min at room temperature. The substrates were put into a 24-well plate and hydrated in PBS (0.5 ml) for 2 h at 37° C. prior to adsorption. The buffer was aspirated and replaced with 1 mg/ml fibrinogen from human plasma (F3879, Sigma-Aldrich, 0.5 ml) or 10% PPP (0.5 ml). Adsorption was allowed to continue at 37° C. for 90 min. Then, the substrates were rinsed five times with PBS and incubated in a bovine serum albumin solution (BSA, A7906, Sigma-Aldrich, 1 mg/ml in PBS) for 90 min at 37° C. to block the areas unoccupied by fibrinogen. The substrates were rinsed with PBS five times again, transferred to new wells, and incubated in a PBS solution (0.5 ml) containing 10 ng/ml horseradish peroxidase (HRP) conjugated anti-fibrinogen (F4200-07C, USBiological, Swampscott, Mass., USA) for 90 min at 37° C. Afterwards, the substrates were rinsed 5 times with PBS and transferred into clean wells, followed by the addition of 0.05 M citrate-phosphate buffer (pH 5.0, 0.5 ml) containing 0.5 mg/ml chromogen of o-phenylenediamine (OPD) and 0.03% hydrogen peroxide. After incubation for 20 min at 37° C., the enzyme-induced color reaction was stopped by adding 1 M H₁₂SO₄ (0.5 ml) to the solution in each well. Finally the absorbance of light intensity at 490 nm was determined by a microplate reader. Negative control experiments without the addition of fibrinogen or 10% PPP were also carried out.

Results and Discussion

Surface Grafting of polySBMA from Silanized Silicon and SNM

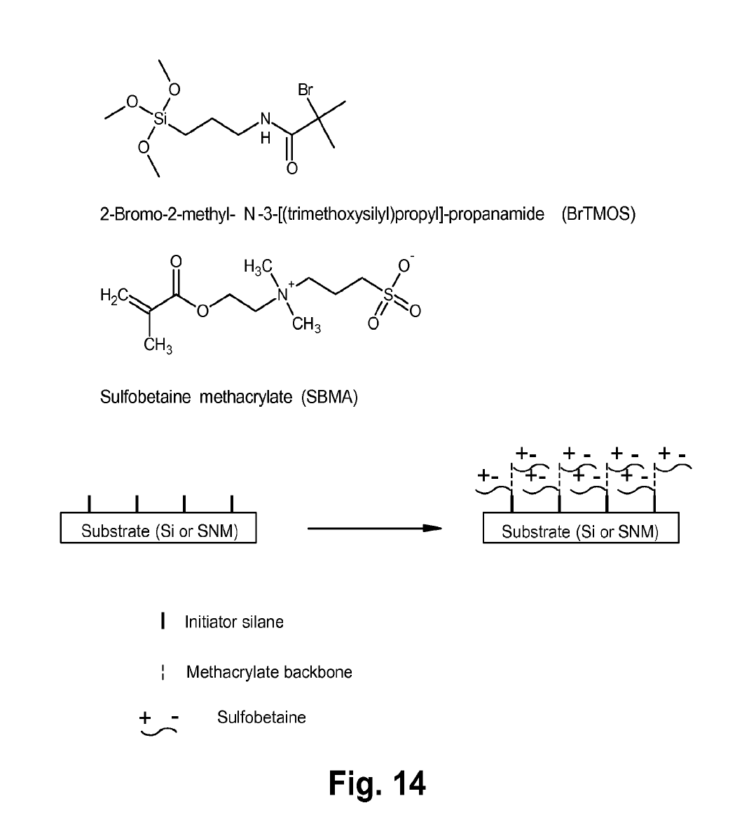

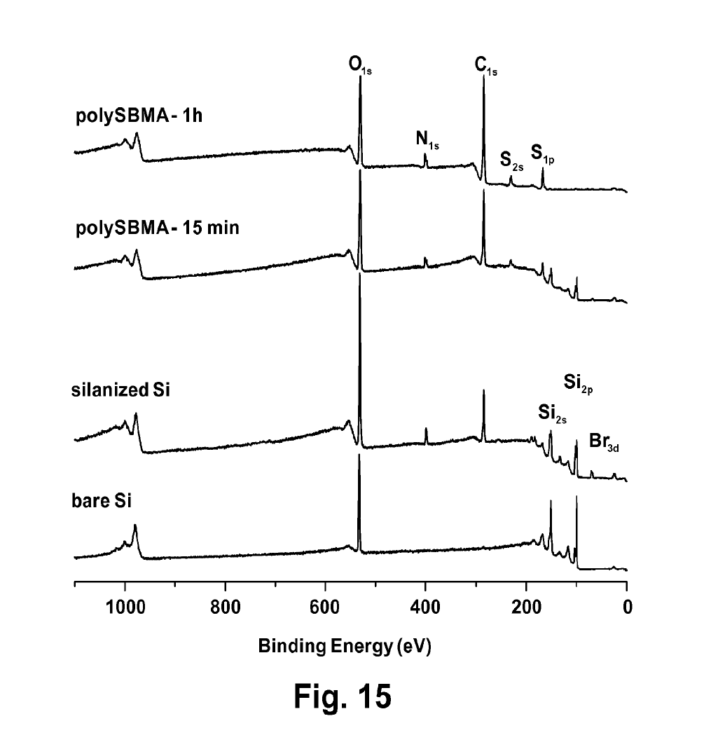

PolySBMA was grafted on silicon surfaces by SI-ATRP as shown schematically in FIG. 14B. First, the initiator of ATRP was immobilized on a silicon surface through silanization. Then, the silanized silicon chips were grafted with polySBMA using SI-ATRP. Compared with the spectrum of bare silicon, new peaks of N and Br appeared on the surface after silanization (FIG. 15). The contact angle of silicon chips after piranha cleaning is about 10 or less, whereas that of silanized surfaces has changed to 41±1.

From the survey scans of the polySBMA grafted surface (FIG. 15), the signals from the silicon components such as Si peaks decreased and the new peaks of sulfur (S2s and S2p) were observed, showing that the surface was covered with a polymer layer. For longer polymerization times, the Si component decreased further on the surface, while surface compositions of C and S increased, and the presence of bromine was no longer detected (Table 1).

TABLE 1
Surface chemical composition (at %) of silicon, BrTMOS-silanized
and polySBMA-grafted silicon surfacs calculated from XPS spectra

| | | | polySBMA | | | Stoichiometric |
|---|---|---|---|---|---|---|
| | Si | BrTMOS | 15 min | 30 mm | 1 h | value |
| O₁ₛ | 52 ± 3 | 38.3 ± 0.6 | 28 ± 1 | 25.3 ± 0.5 | 24.7 ± 0.1 | 27.7 |
| C₁ₛ | 6 ± 1 | 31 ± 1 | 51 ± 2 | 62.3 ± 0.6 | 64.8 ± 0.1 | 61.1 |
| Si₂ₚ | 42 ± 1 | 24 ± 2 | 12 ± 2 | 2.4 ± 0.3 | 0.4 ± 0.3 | — |
| N₁ₛ | | 5.5 ± 0.2 | 4.8 ± 0.3 | 5.6 ± 0.3 | 5.4 ± 0.1 | 5.6 |
| S₂ₚ | | — | 3.8 ± 0.3 | 4.4 ± 0.2 | 4.7 ± 0.1 | 5.6 |
| Br_{3d} | | 1.2 ± 0.1 | 0.2 ± 0.1 | — | — | — |

After 1 h polymerization, the mol ratio of [N]/[S] was 1.1, as estimated by XPS, which is in good agreement with the stoichiometric value of the bulk polymer of SBMA (i.e., 1). The water contact angle on polySBMA is about 10±1° for all polySBMA with a polymerization time less than 1 h, which is consistent with a previous report (Azzaroni O et al., Angew. Chem. Int. Edn 45, 1770, 2006).

Table 2 lists the data from XPS survey scans for SNM and SNM grafted with polySBMA for 10 min on both sides. After SI-ATRP, the composition of carbon was increased, the composition of silicon was decreased, and a small amount of nitrogen and bromine appeared on both sides of SNM. These data indicate that polySBMA was grafted onto the SNM surfaces following the same surface modification strategies for single crystal non-porous silicon.

TABLE 2
Surface atomic compositions (at %) of SNM
and polySBMA-grafted SNM measured by XPS

| | |
|---|---|
| SNM | polySBMA-SNM |

| | Front side | Back side | Front side | Back side |
|---|---|---|---|---|
| O₁ₛ | 44.6 | 49.6 | 27.7 | 27.4 |
| C₁ₛ | 8.5 | 11.8 | 49.6 | 50.1 |
| Si₂ₚ | 46.9 | 38.6 | 14.1 | 13.2 |
| N₁ₛ | | | 4.6 | 5.2 |
| S₂ₚ | | | 3.6 | 4.0 |
| Br_{3d} | | | 0.3 | 0.2 |

Thickness of polySBMA as a Function of Polymerization Time

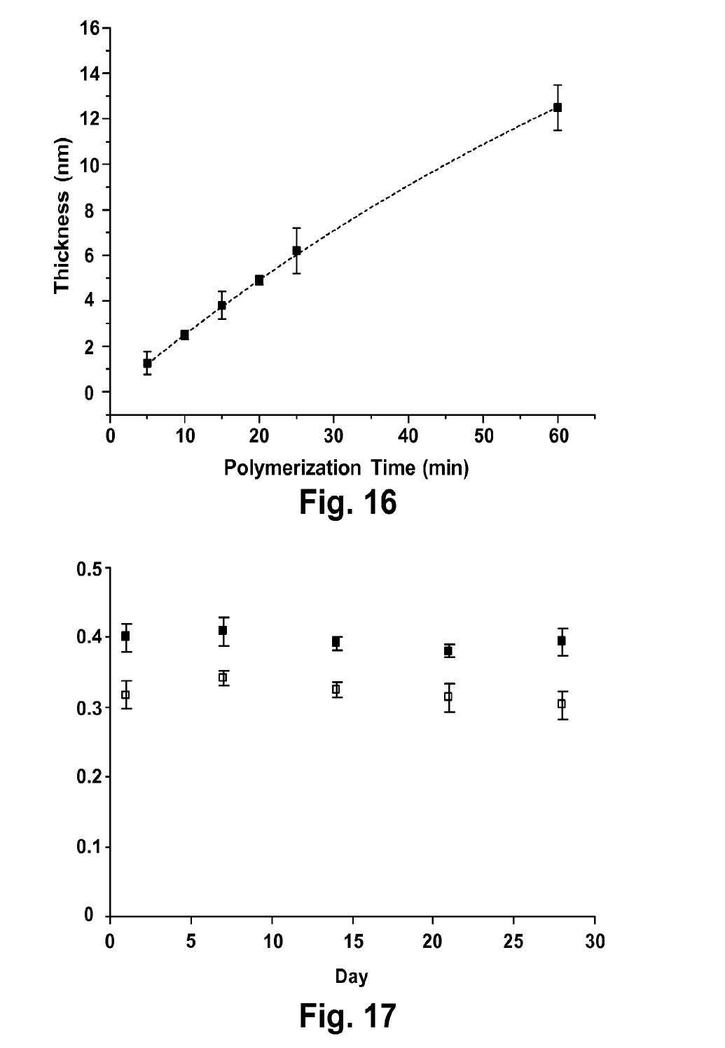

The thickness of polySBMA needs to be controlled precisely in order to coat the silicon nanopore membrane without occluding the nanopores. To guide the design of experimental polymerization conditions, an initial set of experiments was performed on silicon substrates to measure the polymer layer growth kinetics. The thickness of the grafted polymer layer, determined by ellipsometry, is plotted against the polymerization time in FIG. 16. There is a linear increase in polySBMA thickness on silicon substrates with polymerization time. The thickness of polySBMA is about 12 nm after 1 h incubation.

The linear variation of the thickness with reaction time is often observed for ‘living’ polymerization at least in the initial stage of chain growth. For surface-initiated ATRP, the growth rate of polymer films frequently decreases with time, likely due to the small amount of initiator tethered to the substrate, which provides too low a concentration of Cu(II) to control the polymerization. Hence, a method of adding Cu(II) complex (e.g., CuBr₂) is often chosen to control the concentration of the deactivating Cu(II) complex during the surface-initiated ATRP process (Matyjaszewski K et al., Macromolecules 32, 8716, 1999; Huang, W X et al., Macromolecules 35, 1175, 2002; Feng W et al., J. Polym. Sci. Polym. Chem. 42, 2931, 2004). In addition, a high concentration of a deactivating Cu(II) complex is necessary. Cheng et al, observed faster chain growth, but much lower grafting densities in polymer films with higher [Cu(I)]/[Cu(II)] ratios (Cheng N et al., Macromolecules 41, 6317, 2008; Cheng N. et al., Macromol. Rapid Commun. 27, 1632, 2006). To form dense polySBMA films on the SNM, a lower [Cu(I)]/[Cu(II)] ratio was chosen such that the activated radical is reversibly deactivated by the Cu(I) complex. As a result, the graft chains grow slowly but more or less simultaneously.

Analysis of polySBMA Stability in PBS

We examined the stability of polySBMA films on silicon when stored in PBS (pH 7.4, 5% CO₂ and 37° C.) for 4-week periods using XPS. FIG. 17 shows the ratios of surface composition of N₁ₛ to Si₂ₚ (solid squares) and that of S₂ₚ to Si₂ₚ (open squares) for polySBMA-coated silicon substrates after exposure to PBS for extended time. There is no statistical difference (P=0.08) in the ratios of surface compositions between freshly prepared samples and those stored in PBS. Our results indicated that the polySBMA thin films were stable in terms of surface compositions measured by XPS in PBS for 4-week periods.

### Hydraulic Permeability Test

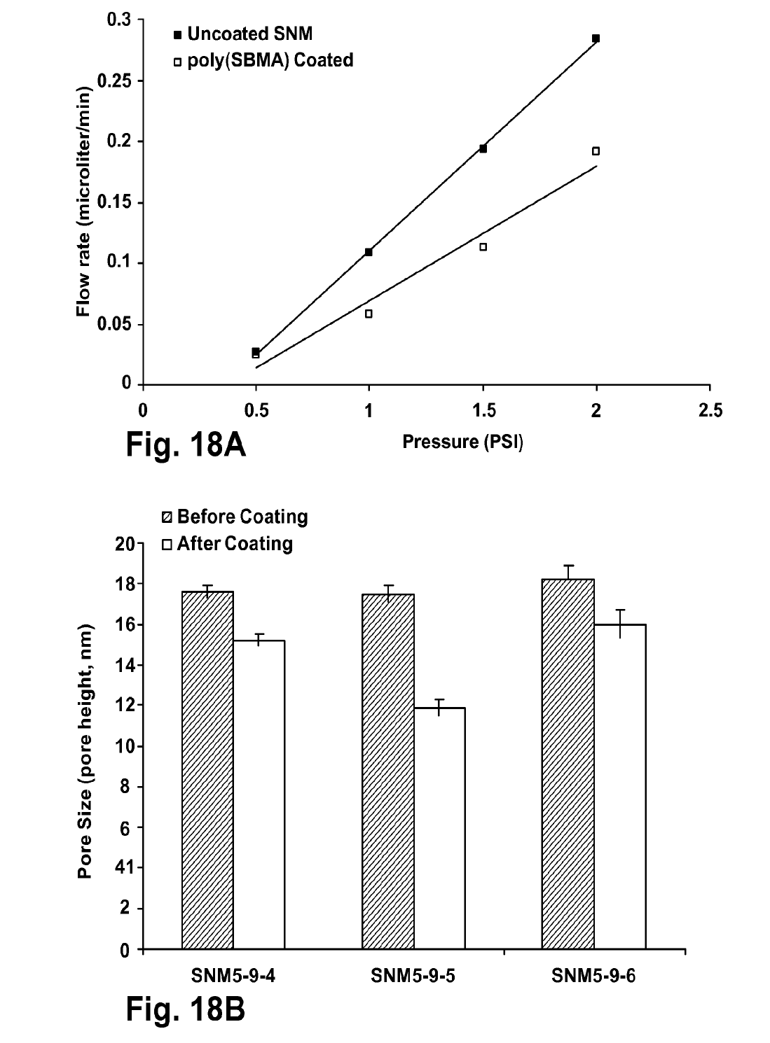

For coating on SNM, 10 min polymerization time was chosen to generate approx. 2.5-nm-thin film coatings on SNM as measured by ellipsometry. Since the measured pressure-flow curves correlated well with theoretical predictions for flow through slit-shaped pipes according to previous reports (Fissell W H et al., Am. J. Physiol. Renal Physiol. 293, F 1209, 2007; Fissell W H et al., J. Am. Soc. Nephrol. 13, 602A, 2002), the equation of Hele-Shaw flows for slit pores can be used to calculate the pore size:

[(Q/ΔP=Wh³/12 μL)],

where W is the long dimension of the slit, h is the short dimension of the slit (or the pore size), L is the thickness of the membrane and, thus, the length of the pore, μ is viscosity, Q is volumetric flow through a single pore and ΔP is transmembrane pressure. The long dimension of the slit is 40 μm and the length of the pore is 4.52 μm as measured by SEM. FIG. 18A shows the measured flow rates versus pressure for one silicon nanopore membrane chip. According to the equation above, the calculated pore heights (or pore sizes) before coating and after coating are 17.6 (R2=0.9994) and 15.2 nm (R2=0.9678), respectively (FIG. 18A). The average film thickness on each side of the pore is approx. 1.2 nm, while that on non-porous surfaces is about 2.5 nm determined by ellipsometry (FIG. 16). The measurements of fluid flow through SNM chips before and after the polySBMA coating were tested for three separate chips. The results are shown in FIG. 18B, in which three of the chips showed a significant reduction (P<0.01) in the calculated pore height (or the pore size). Our results suggested that the polySBMA is distributed within the pore and not exclusively at the surface.

### Reduction of Fibrinogen Adsorption on SNM

Non-specific adsorption of proteins from blood (e.g., fibrinogen), can cause fouling, and initiate platelet adhesion, aggregation and thrombosis, leading to device failure. Hence, fibrinogen binding is often used as a method to determine hemocompatibility. Detection of fibrinogen on the surface has been performed by a variety of techniques, most notably by labeling the molecule with 1251 or by using an ELISA technique (Slack S M et al., J. Biomater. Sci. Polymer Edn 3, 49, 1991; Brash J L et al., Thromb. Haemost. 51, 326, 1984). The sensitivity of the ELISA method has been found to be equivalent to that of the radio labeled method (Slack S M et al., cited above). Fibrinogen adsorption measured with the direct ELISA methodology consists of the adsorption of the desired protein (antigen) to a substrate followed by attachment of an antibody-enzyme complex to the bound antigen. The bulk protein solution is rinsed away and a chromogenic substrate for the enzyme is introduced. The intensity of the color change resulting from the enzymatic conversion of the substrate was measured as absorbance or optical density, which is proportional to the amount of protein adsorbed on the surface.

Fibrinogen adsorption on polySBMA-grafted silicon with different film thickness was determined by a direct ELISA method. The average optical density values measured at 490 nm for the untreated silicon and initiator BrTMOS-coated silicon are 0.7±0.1 and 0.6±0.1, respectively. However, the optical density values dropped to 0.04±0.02 for polySBMA-coated chips, which is similar to the background signal detected by the negative control experiments (0.06±0.02). Results show that fibrinogen adsorption in optical density is independent of the film thickness of grafted polySBMA (2-20 nm by ellipsometry).

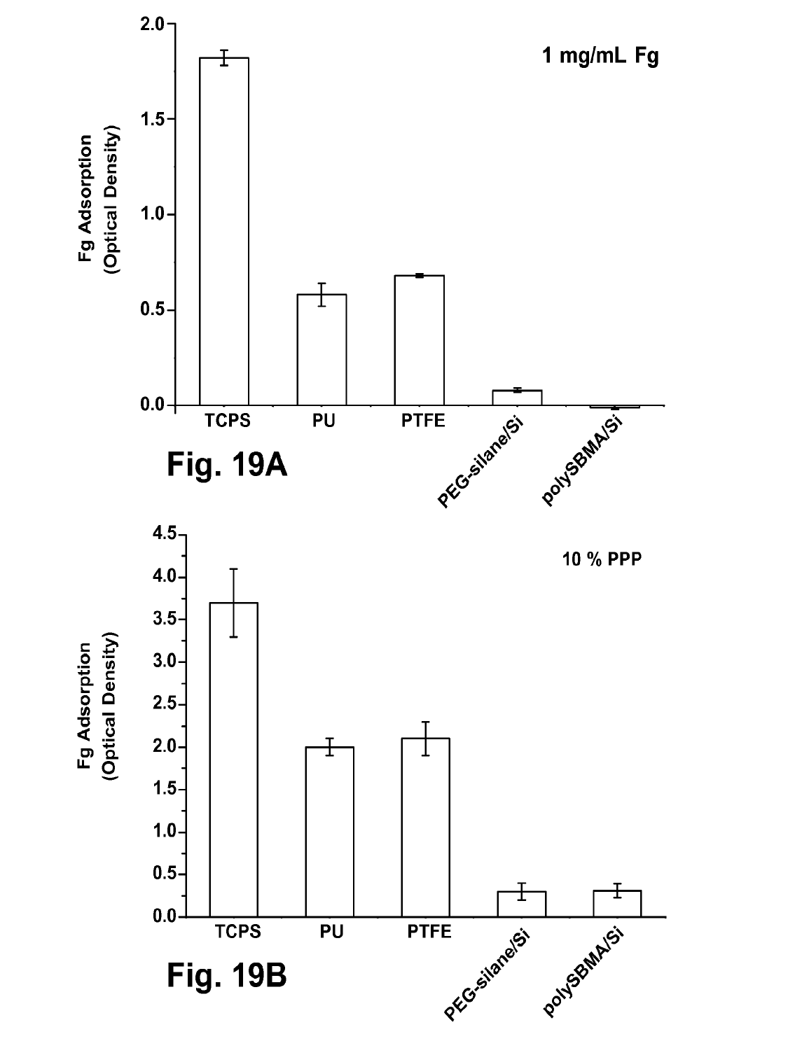

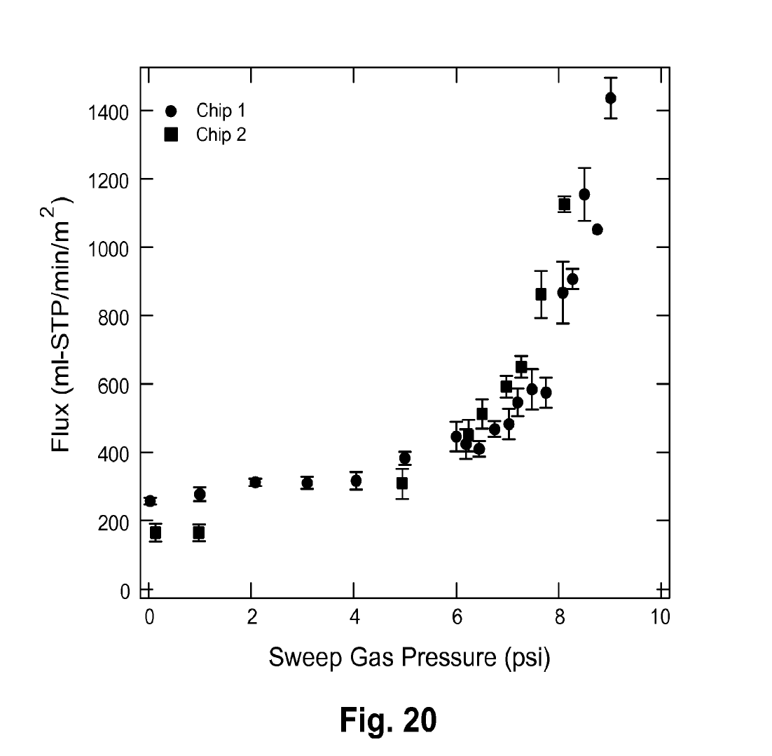

Fibrinogen adsorption from 10% PPP, as well as from 1 mg/ml fibrinogen solution was also performed. For comparison, protein adsorption on tissue-culture polystyrene (TCPS), two frequently used polymer biomaterials, polyurethane (PU, Precision Urethane) and polytetrafluoroethylene (PTFE, Enflo, Bristol, Conn., USA) and self-assembled 2-[methoxy(polyethyleneoxy)propyl]trimethoxysilane (PEG, Gelest, Morrisville, Pa., USA) were tested. FIGS. 19A-B show the adsorption of fibrinogen from 1 mg/ml fibrinogen solution and 10% PPP on several surfaces. It is found that the polySBMA-coated substrates had a lower protein adsorption than other tested polymer surfaces. We noticed that fibrinogen adsorption from 10% PPP is different than from single fibrinogen solution. The fibrinogen adsorption on the surfaces may be influenced by the coexisting proteins such as serum albumin in the plasma solution.

For polySBMA to be used in practical applications as a non-fouling coating, its long-term stability in a biological environment is crucial. In this work, the modified surfaces were incubated in PBS (pH 7.4, 5% CO₂ and 37° C.) over an extended period of time to study their stability in aqueous solution. After incubation in PBS for 7, 21 and 28 days each sample was analyzed using ELISA as previously described. Table 3 gives the amounts of fibrinogen adsorbed on polySBMA- and PEG-silane-coated membranes relative to that on TCPS over an extended period of time.

TABLE 3
Maintenance of Fg resistance over time for polySBMA
and PEG-silane determined by relative Fg adsorption
with respect to that on TCPS using an ELISA method

Relative Fg adsorption

| | From 1 mg/ml Fg | From 10% PPP |
|---|---|---|
| polySBMA/Si | | |
| Day 1 | −0.01 ± 0.01 | 0.08 ± 0.04 |
| Day 7 | 0.01 ± 0.02 | 0.08 ± 0.04 |
| Day 21 | −0.01 ± 0.02 | 0.09 ± 0.02 |
| Day 28 | −0.01 ± 0.01 | 0.09 ± 0.03 |
| PEG-Silane/Si | | |
| Day 1 | 0.04 ± 0.01 | 0.08 ± 0.03 |
| Day 7 | 0.08 ± 0.02 | 0.2 ± 0.1 |
| Day 21 | 0.2 ± 0.1 | 0.3 ± 0.1 |
| Day 28 | 0.5 ± 0.1 | 0.3 ± 0.1 |

The results show that the fibrinogen repellent property of polySBMA thin films was maintained under in vitro simulated physiological conditions over 28 days, whereas PEG-silane-coated silicon substrates adsorbed a significant amount of fibrinogen after being stored in PBS for 28 days. In addition, analysis of the surfaces by XPS indicated that the polySBMA films remained stable (in terms of the surface chemical composition of each atom) in PBS. This result demonstrates that surface grafted polySBMA on silicon can reduce fibrinogen adsorption and retain its repulsive properties for at least 4 weeks in solution, indicating that zwitterionic polymers may offer a good alternative to PEG-based materials for resisting nonspecific protein adsorption.

Hydrophilic PEG-based polymers, zwitterionic polymers and polymers incorporating oligosaccharide moieties are inherently anti-biofouling in nature. Significant efforts have been directed toward developing a fundamental understanding of their anti-biofouling mechanisms. Although both experimental and theoretical studies suggest that the formation of a hydration layer near a hydrophilic surface is a general basis for protein resistance, discussion regarding hydration versus steric repulsion mechanisms for antifouling activity continues. Similar to PEG-based materials, zwitterionic groups also have a strong influence on interfacial water molecules. Hydrophilic PEG chains form a hydration layer through hydrogen bonds whereas zwitterionic chains through both ionic solvation and hydrogen bonds. Thus, zwitterionic groups strongly hydrated through ionic solvation may be the key to their non-fouling properties.

From the above description of the invention, those skilled in the art will perceive improvements, changes and modifications. Such improvements, changes, and modifications are within the skill of the art and are intended to be covered by the appended claims.

Having described the invention, the following is claimed:

1. A silicon nanoporous membrane for oxygenating and/or removing carbon dioxide from blood, said nanoporous membrane comprising:
a first major surface for contacting a gas;
a second major surface for contacting blood and being oppositely disposed from said first major surface, said first and second major surfaces defining a membrane thickness; and
a plurality of pores extending between said first and second major surfaces, each of said pores being defined by a length, a width, and a height, each of said pores being separated by a uniform interpore distance

2. The nanoporous membrane of claim 1, wherein each of said pores has the same bubble point to prevent or mitigate membrane failure through pore wetting.

3. The nanoporous membrane of claim 1, wherein said nanoporous membrane thickness is about 0.1 micrometer to about 50 micrometers.

4. The nanoporous membrane of claim 3, wherein said nanoporous membrane has a flattened, sheet-like configuration.

5. The nanoporous membrane of claim 1, wherein each of said pores is slit-shaped.

6. The nanoporous membrane of claim 1, wherein each of said pores has a symmetrical cross-sectional profile.

7. The nanoporous membrane of claim 6, wherein each of said pores has a rectangular cross-sectional profile.

8. The nanoporous membrane of claim 1, wherein each of said pores has an asymmetrical cross-sectional profile.

9. The nanoporous membrane of claim 8, wherein each of said pores has a tapered cross-sectional profile.

10. The nanoporous membrane of claim 1, wherein said length of each of said pores is about 0.1 micrometers to about 1000 micrometers.

11. The nanoporous membrane of claim 1, wherein said width of each of said pores is at least about 0.5 nanometers.

12. The nanoporous membrane of claim 1, wherein said interpore distance is less than about 3 micrometers.

13. The nanoporous membrane of claim 1, wherein at least a portion of said nanoporous membrane is treated with one or more biocompatible materials to prevent or minimize biofouling.

14. The nanoporous membrane of claim 13, wherein at least a portion of said membrane is treated with a biocompatible material selected from the group consisting of poly(sulfobetaine methacrylate) (polySBMA), PEG and PVAm.

15. A portable extracorporeal respiratory gas exchanger comprising:
a silicon nanoporous membrane comprising:
a first major surface for contacting a gas;
a second major surface for contacting blood and being oppositely disposed from said first major surface, said first and second major surfaces defining a membrane thickness; and
a plurality of pores extending between said first and second major surfaces, each of said pores being defined by a length, a width, and a height, each of said pores being separated by a uniform interpore distance;
a housing containing said nanoporous membrane;
a first fluid passageway configured to receive blood from a subject's vasculature and deliver blood to said second major surface of said nanoporous membrane;
a gas passageway configured to deliver the gas to said first major surface of said nanoporous membrane; and
a second fluid passageway configured to remove oxygenated blood from said housing and deliver the oxygenated blood to the vasculature of the subject.

16. The extracorporeal respiratory gas exchanger of claim 15, wherein said extracorporeal respiratory gas exchanger is pumpless.

17. The extracorporeal respiratory gas exchanger of claim 15 further including a second gas passageway configured to remove at least some of the gas from said housing.

18. The extracorporeal respiratory gas exchanger of claim 15, wherein the blood-gas phase interface is maintained at said second major surface of said nanoporous membrane during operation of the said extracorporeal respiratory gas exchanger.

19. The extracorporeal respiratory gas exchanger of claim 15, wherein each of said pores has the same bubble point to prevent or mitigate membrane failure through pore wetting.

20. The extracorporeal respiratory gas exchanger of claim 15, wherein said membrane thickness is about 0.1 micrometer to about 50 micrometers.

21. The extracorporeal respiratory gas exchanger of claim 15, wherein said nanoporous membrane has a flattened, sheet-like configuration.

22. The extracorporeal respiratory gas exchanger of claim 15, wherein each of said pores is slit-shaped.

23. The extracorporeal respiratory gas exchanger of claim 15, wherein each of said pores has a symmetrical cross-sectional profile.

24. The extracorporeal respiratory gas exchanger of claim 23, wherein each of said pores has a rectangular cross-sectional profile.

25. The extracorporeal respiratory gas exchanger of claim 15, wherein each of said pores has an asymmetrical cross-sectional profile.

26. The extracorporeal respiratory gas exchanger of claim 25, wherein each of said pores has a tapered cross-sectional profile.

27. The extracorporeal respiratory gas exchanger of claim 15, wherein said length of each of said pores is about 0.1 micrometers to about 1000 micrometers.

28. The extracorporeal respiratory gas exchanger of claim 15, wherein said width of each of said pores is at least about 5 nanometers.

29. The extracorporeal respiratory gas exchanger of claim 15, wherein said interpore distance is less than about 3 micrometers.

30. The extracorporeal respiratory gas exchanger of claim 15, wherein at least a portion of said nanoporous membrane is treated with one or more biocompatible materials to prevent or minimize biofouling.

31. The extracorporeal respiratory gas exchanger of claim 30, wherein at least a portion of said nanoporous membrane is treated with a biocompatible material selected from the group consisting of polySBMA, PEG and PVAm.

32. A method for treating a respiratory disorder in a subject, said method comprising the steps of:
providing a portable extracorporeal respiratory gas exchanger, the extracorporeal respiratory gas exchanger comprising a silicon nanoporous membrane, a housing, a first fluid passageway, a second fluid passageway, and a gas passageway, the nanoporous membrane comprising oppositely disposed first and second major surfaces that define a membrane thickness and a plurality of pores extending between the first and second major surfaces, each of the pores being defined by a length, a width, and a height, each of the pores being separated by a uniform interpore distance, the housing containing the nanoporous membrane;
connecting a vein and artery of the subject to the first and second fluid passageways, respectively;
infusing a gas into the gas passageway at a pressure sufficient to ensure that the blood-gas phase interface is maintained at the second major surface of the nanoporous membrane;
whereby blood flowing through the extracorporeal respiratory gas exchanger is oxygenated and delivered to the vasculature of the subject via the second fluid passageway.
